(19) United States
(12) Patent Application Publication
(10) Pub. No.: US 2021/0161556 A1
(43) Pub. Date: Jun. 3, 2021

(54) CANNULA, CANNULA SYSTEM, AND MANIPULATOR

(71) Applicant: OLYMPUS CORPORATION, Tokyo (JP)

(72) Inventor: Hiromu IKEDA, Tokyo (JP)

(73) Assignee: OLYMPUS CORPORATION, Tokyo (JP)

(21) Appl. No.: 17/170,007

(22) Filed: Feb. 8, 2021

Related U.S. Application Data
(63) Continuation of application No. PCT/JP2018/030409, filed on Aug. 16, 2018.

(51) Int. Cl. A61B 17/34

(52) U.S. Cl. CPC A61B 17/3421; A61B 2017/3454

(57) ABSTRACT

A cannula includes a first tubular member having a first longitudinal axis, a second tubular member having a second longitudinal axis, a connector that connects the first and second tubular members in series, and a rear bearing that supports the second tubular member in a three-dimensionally pivotable manner around an axis intersecting the second longitudinal axis. The first tubular member has a first through-hole into which a flexible and elongated medical treatment device is insertable, and is supported by a trocar in a state where the first tubular member extends through the trocar attached to a body wall of a patient. The second tubular member has a second through-hole into which the medical treatment device is insertable. The connector connects the first and second tubular members in a pivotable manner around an axis orthogonal to a plane including the first longitudinal axis and the second longitudinal axis.

### CROSS-REFERENCE TO RELATED APPLICATIONS

This is a continuation of International Application PCT/JP2018/030409 which is hereby incorporated by reference herein in its entirety.

### TECHNICAL FIELD

The present invention relates to cannulas, cannula systems, and manipulators.

### BACKGROUND ART

A known medical manipulator is equipped with a medical treatment device, such as surgical forceps having a distal-end treatment section and a proximal-end operating section (e.g., see Patent Literature 1 and Patent Literature 2). The medical manipulator described in Patent Literature 1 moves the medical treatment device about a trocar serving as a fulcrum attached to the body wall of a patient, so that the proximal-end operating section disposed outside the body cavity and the distal-end treatment section to be inserted into the body cavity move in directions opposite to each other. This is problematic in terms of poor operability of the medical treatment device, thus requiring a highly skilled surgeon.

In contrast, the manipulator described in Patent Literature 2 is for an endoscopic surgical device and transmits the movement of the proximal-end operating section to the distal-end treatment section by means of a pulley or a pantograph, thereby synchronizing the movement of the proximal-end operating section at the hands of the surgeon with the movement of the distal-end treatment section within the body cavity.

### CITATION LIST

### Patent Literature

### {PTL 1}

Japanese Unexamined Patent Application, Publication No. 2006-150105

### {PTL 2}

Japanese Unexamined Patent Application, Publication No. 2009-018027

### SUMMARY OF INVENTION

A first aspect of the present invention provides a cannula including a first tubular member having a first longitudinal axis, a second tubular member having a second longitudinal axis, a connector that connects the first tubular member and the second tubular member in series, and a rear bearing that supports the second tubular member in a three-dimensionally pivotable manner around an axis intersecting the second longitudinal axis. The first tubular member has a first through-hole into which a flexible and elongated medical treatment device is insertable, and is supported by a trocar in a state where the first tubular member extends through the trocar attached to a body wall of a patient. The second tubular member has a second through-hole into which the medical treatment device is insertable. The connector connects the first tubular member and the second tubular member in a pivotable manner around an axis orthogonal to a plane including the first longitudinal axis and the second longitudinal axis.

A second aspect of the present invention provides a cannula including a first tubular member having a first longitudinal axis, a second tubular member having a second longitudinal axis, a connection mechanism that connects the first tubular member and the second tubular member in series, a front bearing that supports the first tubular member in a three-dimensionally pivotable manner around an axis intersecting the first longitudinal axis, a rear bearing that supports the second tubular member in a three-dimensionally pivotable manner around an axis intersecting the second longitudinal axis, and a support member configured to accommodate the connection mechanism therein and that secures a relative position between the front bearing and the rear bearing with a distance therebetween. The first tubular member has a first through-hole into which a flexible and elongated medical treatment device is insertable. The second tubular member has a second through-hole into which the medical treatment device is insertable. The connection mechanism connects the first tubular member and the second tubular member in a pivotable manner around an axis orthogonal to a plane including the first longitudinal axis and the second longitudinal axis.

### BRIEF DESCRIPTION OF DRAWINGS

FIG. 1 illustrates the overall configuration of a cannula and a manipulator according to a first embodiment of the present invention.

FIG. 2A illustrates the overall configuration of a medical treatment device in FIG. 1.

FIG. 2B illustrates a state where a gripping section in FIG. 2A is bent.

FIG. 3 is an enlarged view of a handle in FIG. 2A.

FIG. 4A illustrates the overall configuration of the cannula in FIG. 1.

FIG. 4B illustrates a state where a first tubular member and a second tubular member of the cannula in FIG. 4A are pivoted.

FIG. 5A is a cross-sectional view of the cannula in FIG. 1.

FIG. 5B is a vertical sectional view of the cannula in FIG. 1.

FIG. 6A illustrates the overall configuration of a connection mechanism in FIG. 4A.

FIG. 6B is a perspective view illustrating a state where the connection mechanism in FIG. 6A is pivoted around a rotation axis.

FIG. 7A illustrates the overall configuration of a front bearing (or a rear bearing) in FIG. 4A.

FIG. 7B is a vertical section view of the front bearing (or the rear bearing) in FIG. 7A.

FIG. 8A illustrates a state where the cannula and the medical treatment device in FIG. 1 are linearly extended.

FIG. 8B illustrates a state where the medical treatment device in FIG. 8A is bent together with the cannula.

FIG. 9A illustrates a state where the medical treatment device is moved forward while the cannula and the medical treatment device in FIG. 1 are linearly extended.

FIG. 9B illustrates a state where the cannula and the medical treatment device in FIG. 9A are moved forward while being bent.

FIG. 10A illustrates a state where a port is attached to the body wall of a patient while extending therethrough.

FIG. 10B illustrates a state where the first tubular member of the cannula is inserted in and supported by the port in FIG. 10A while extending therethrough.

FIG. 100 illustrates a state where the gripping section of the medical treatment device is inserted in a body cavity via the cannula in FIG. 10B.

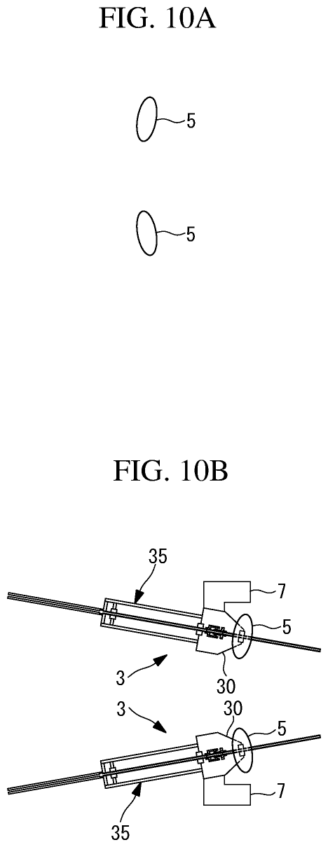

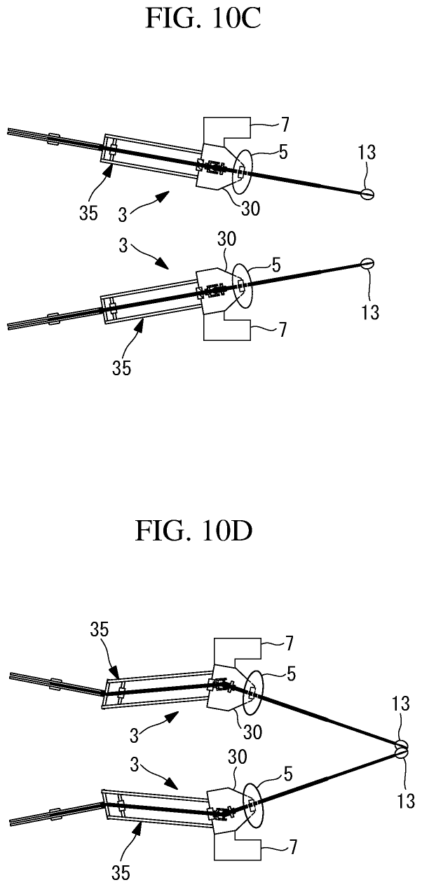

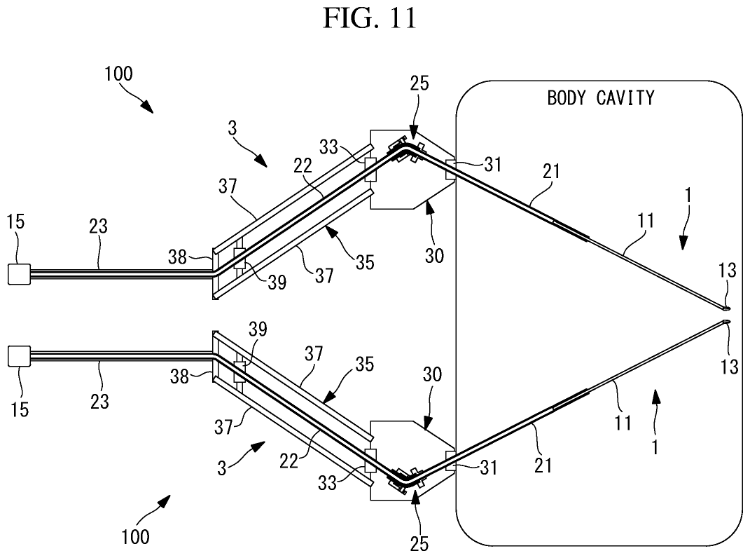

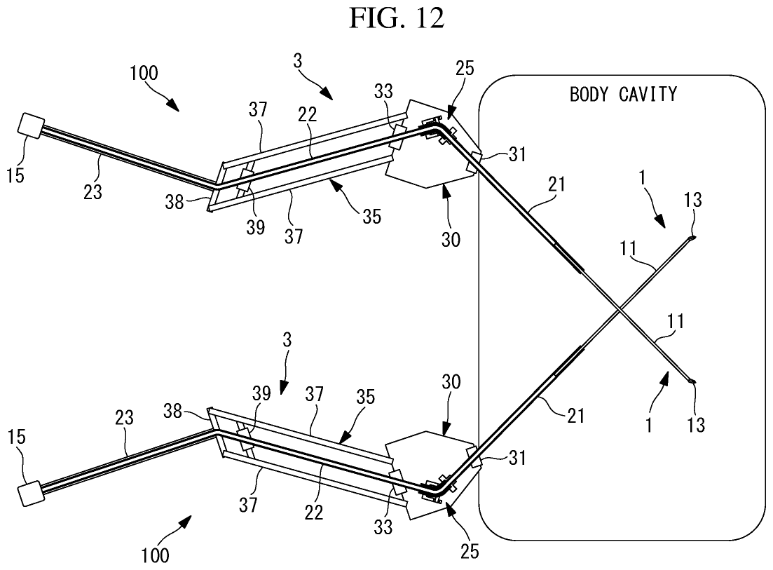

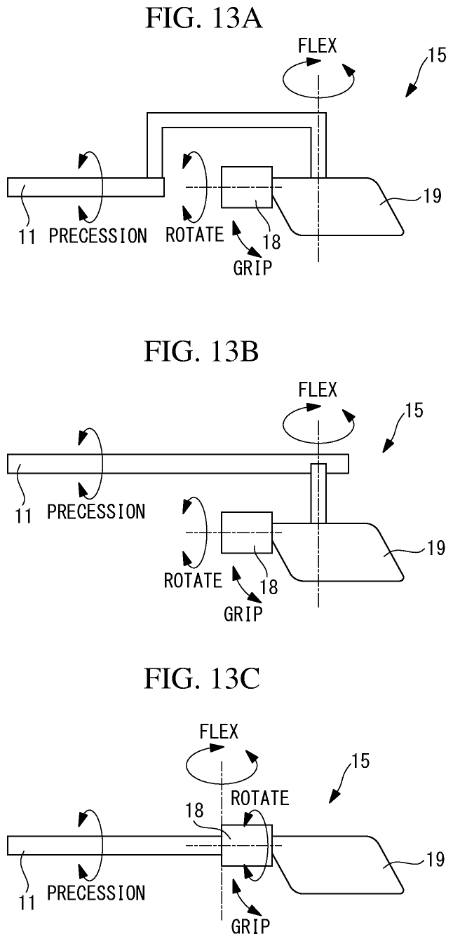

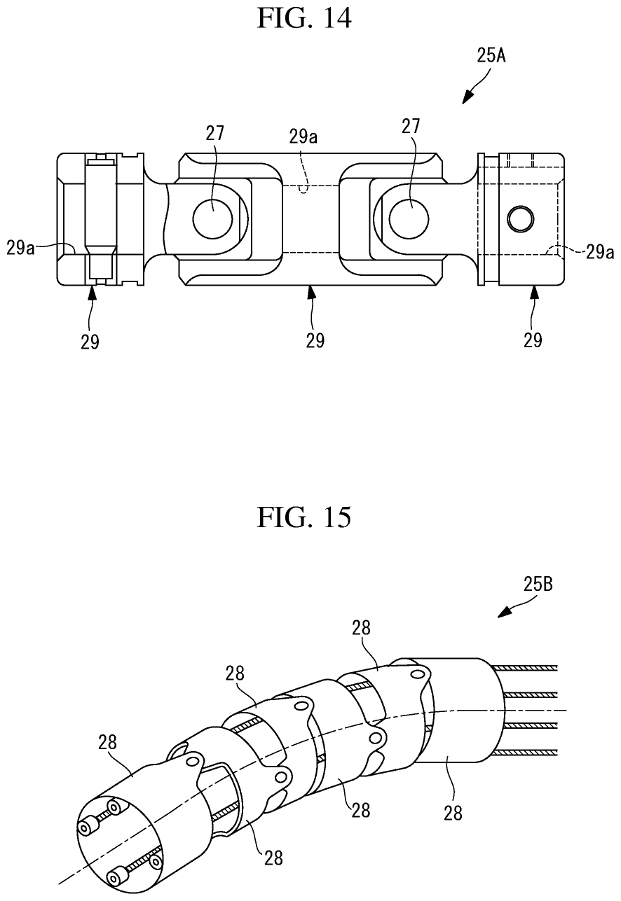

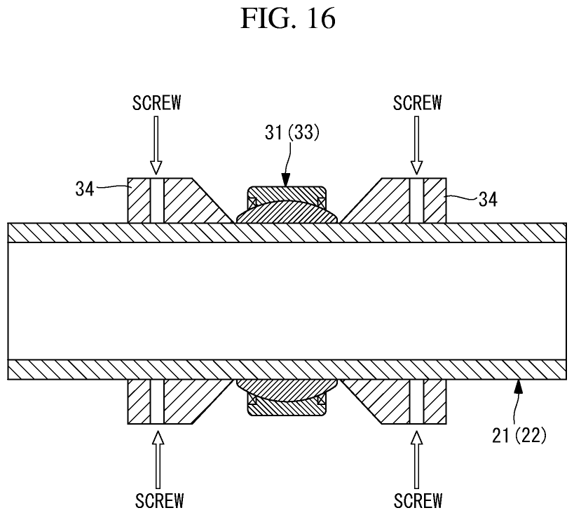

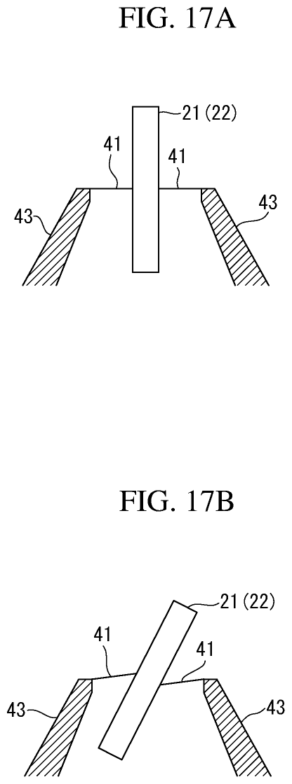

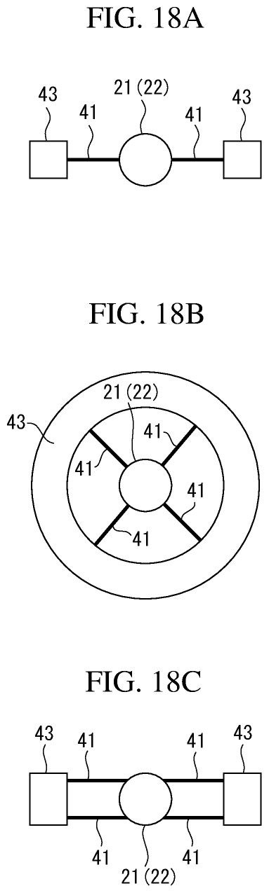

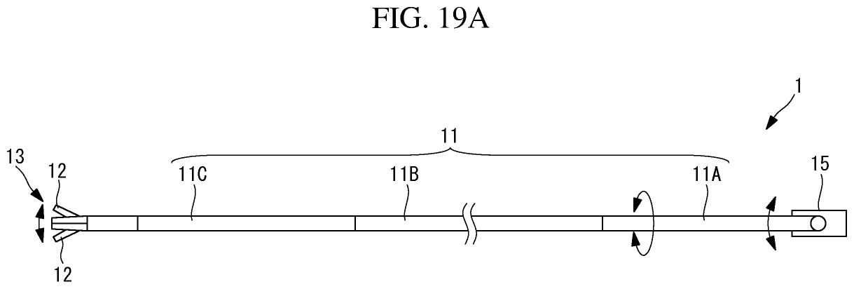

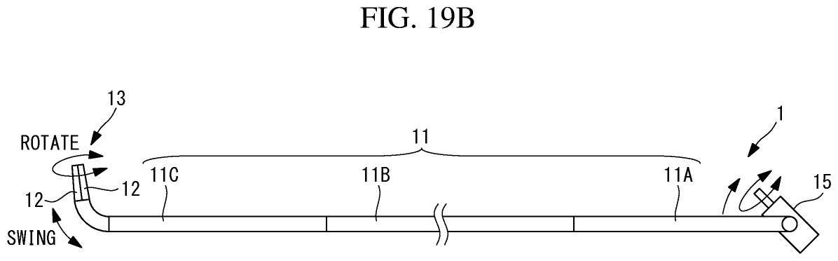

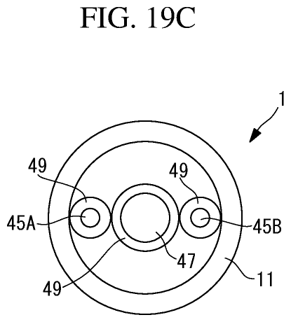

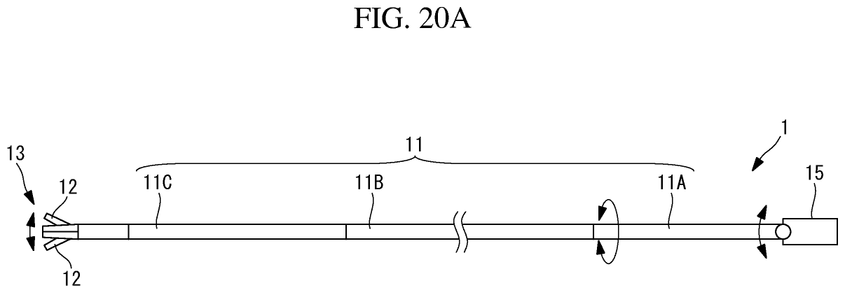

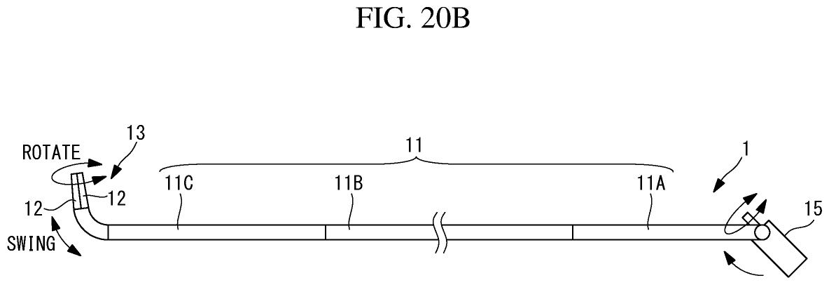

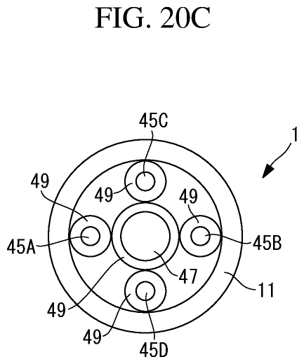

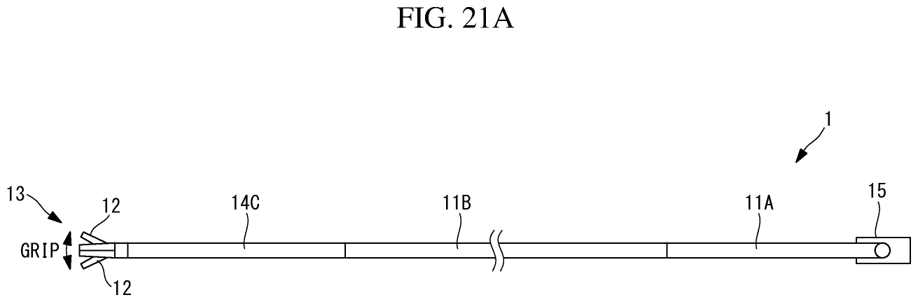

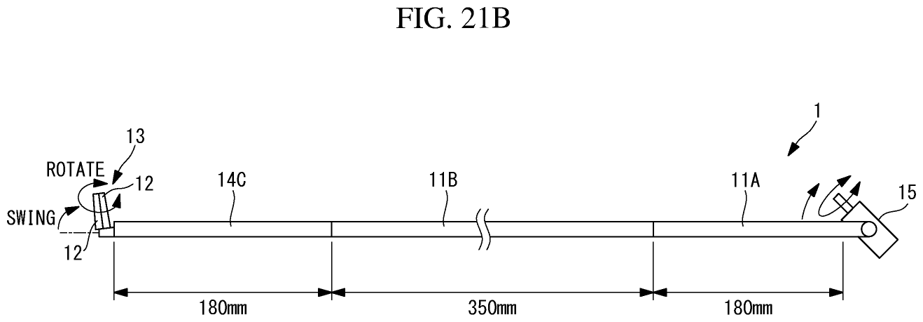

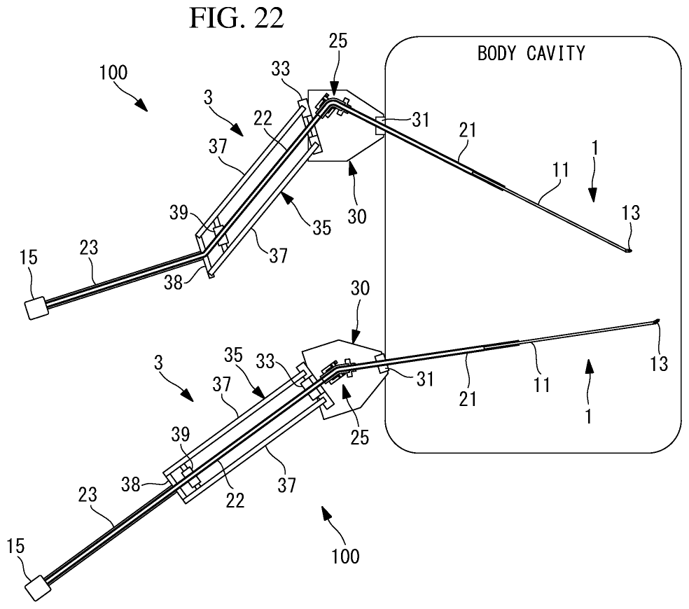

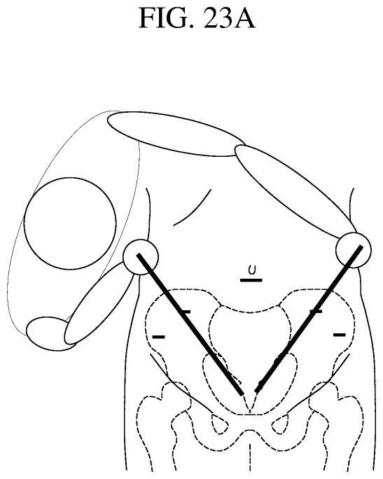

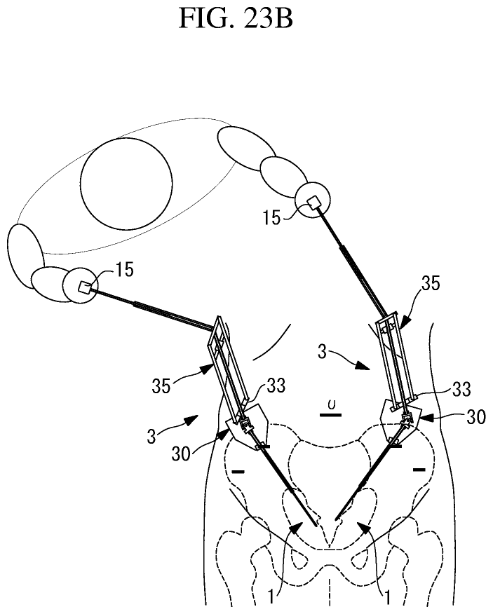

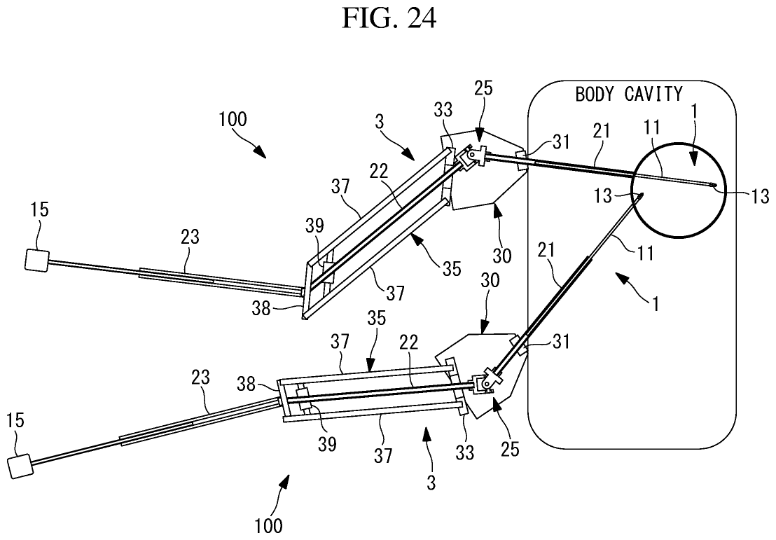

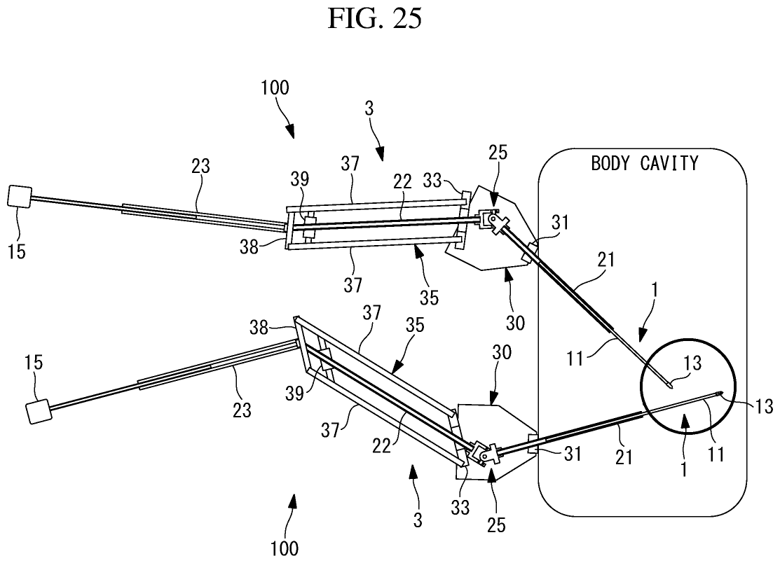

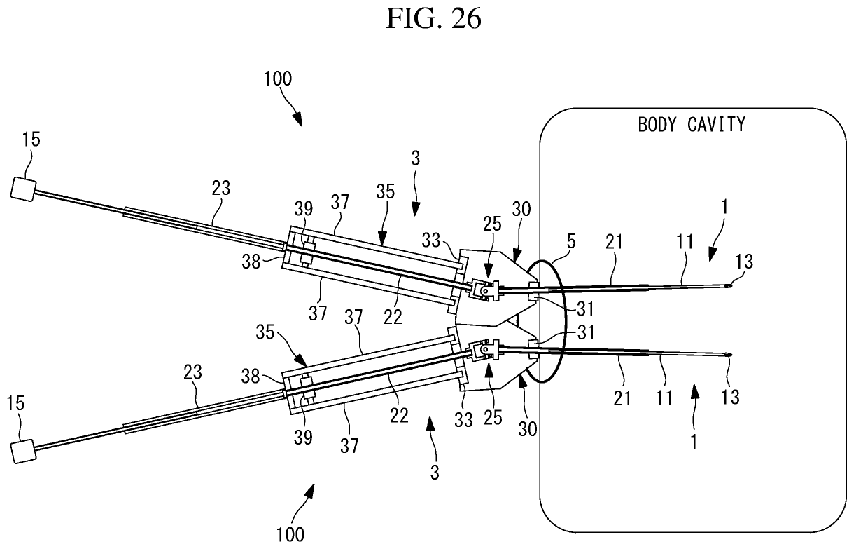

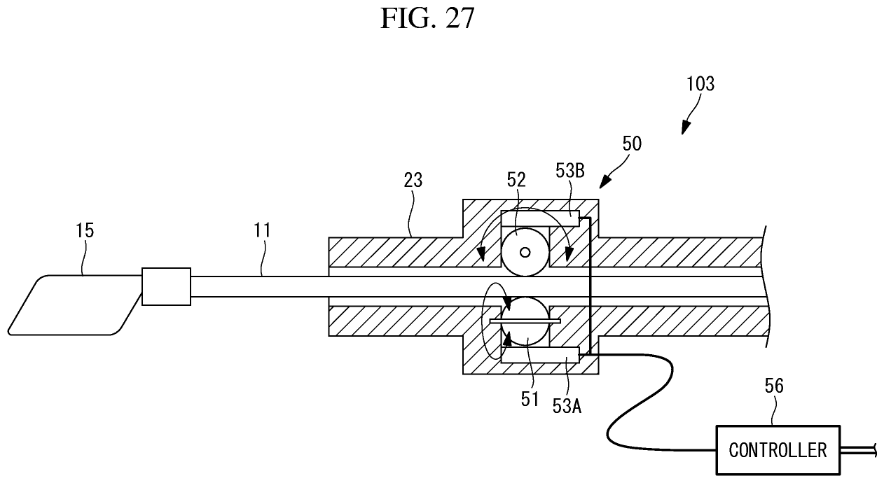

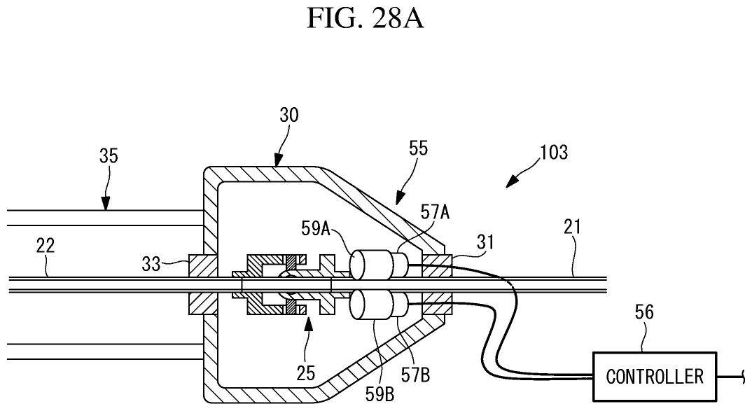

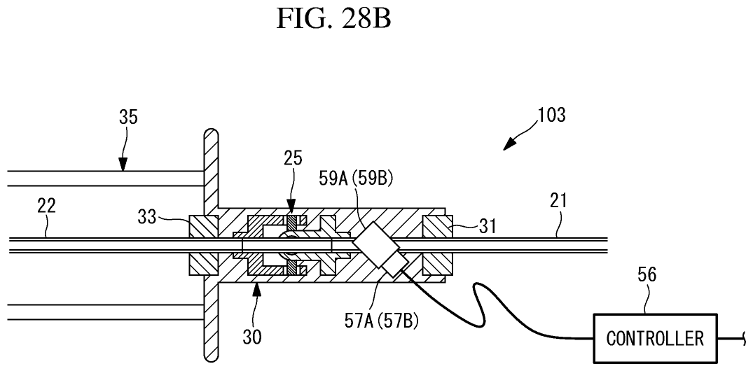

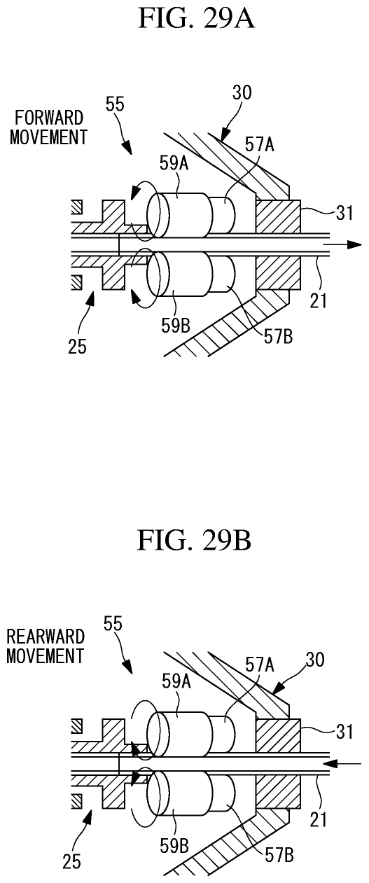

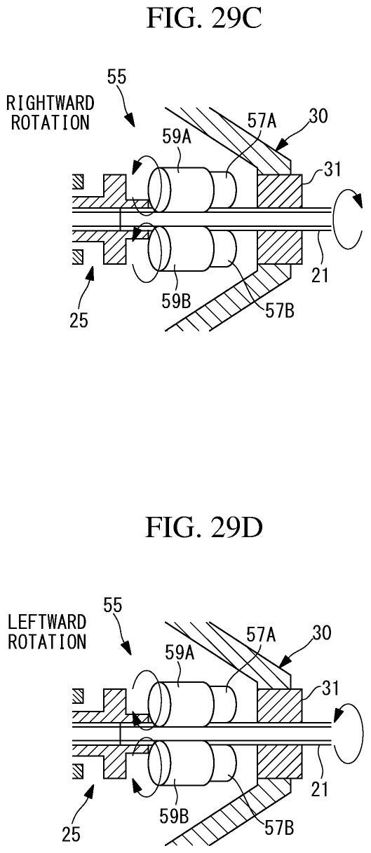

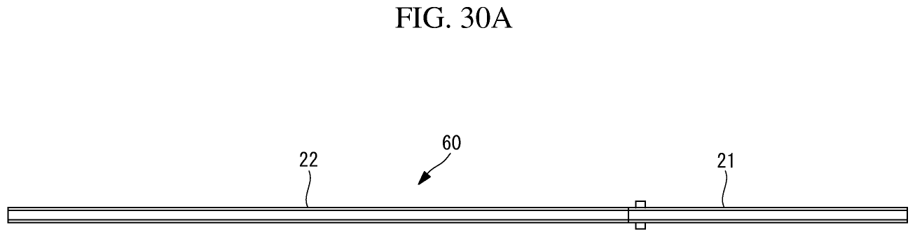

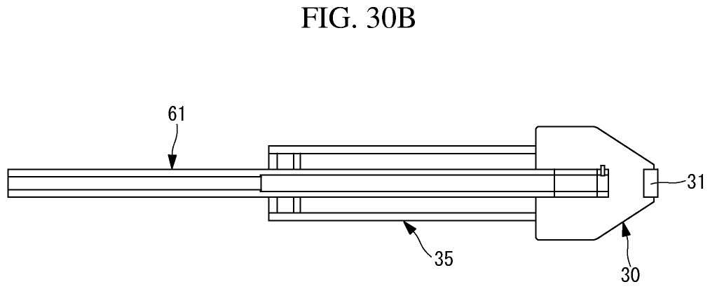

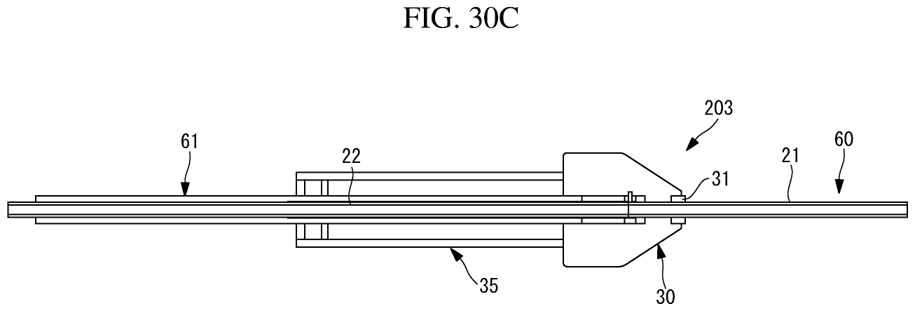

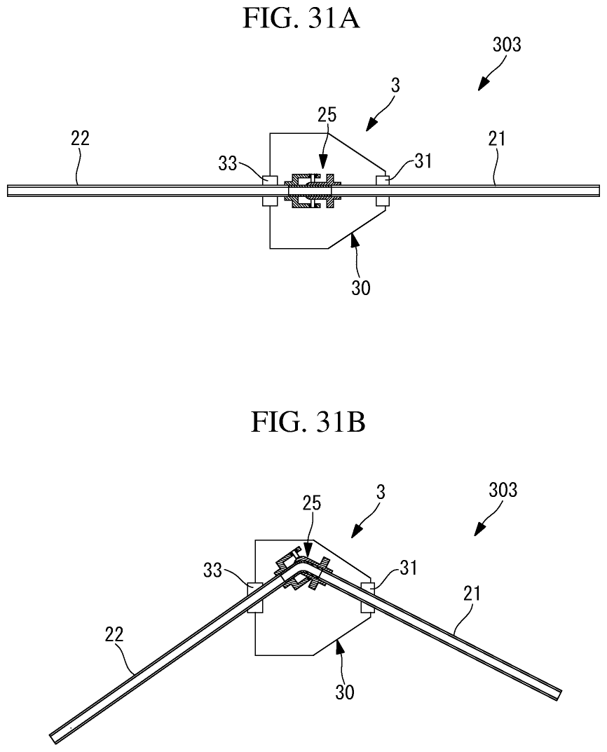

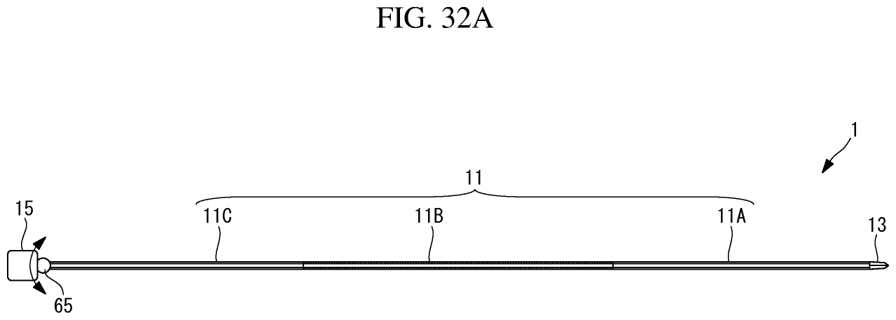

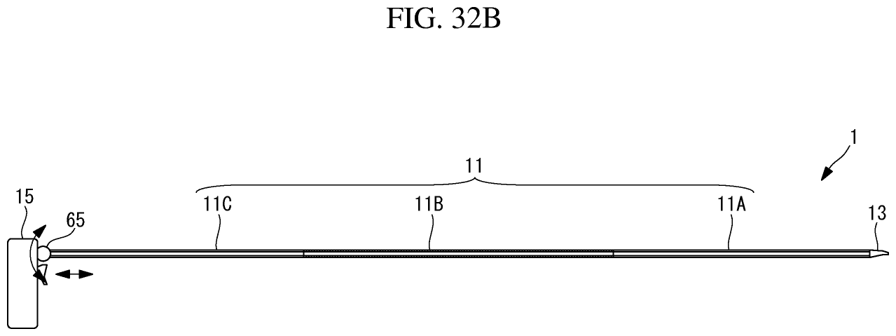

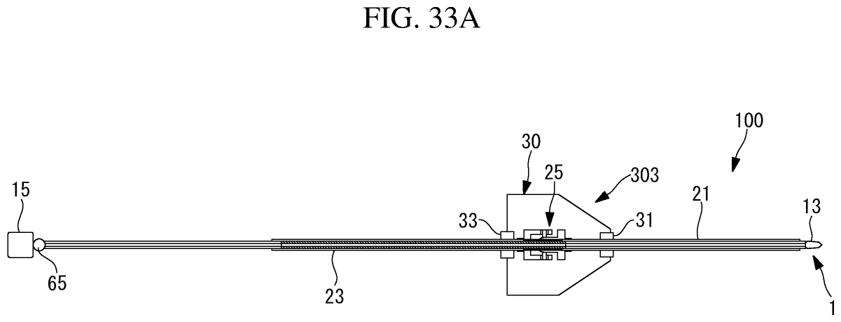

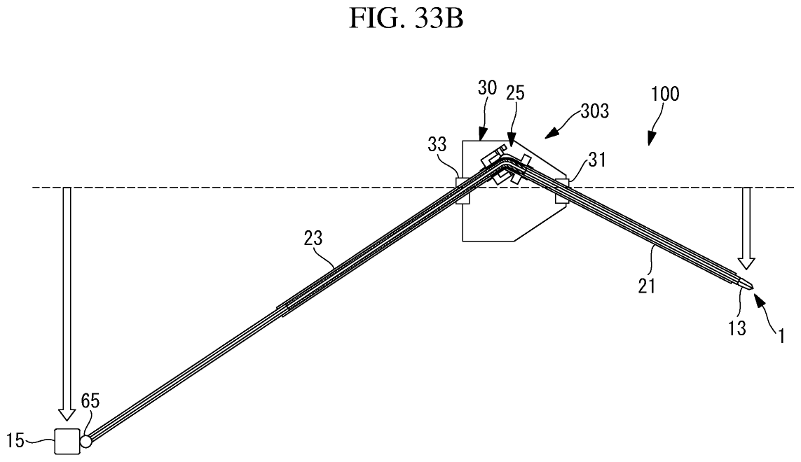

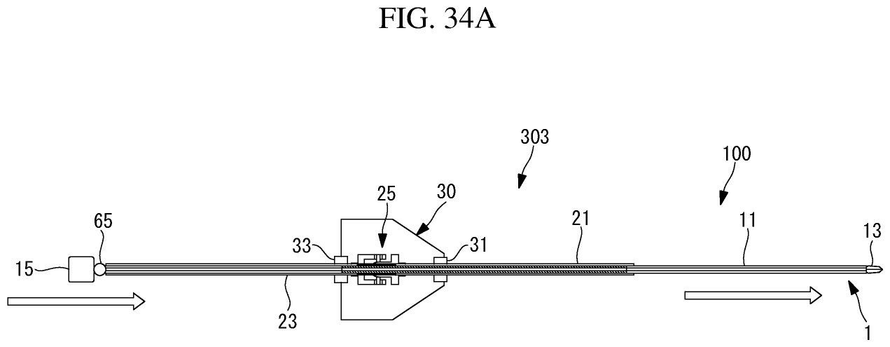

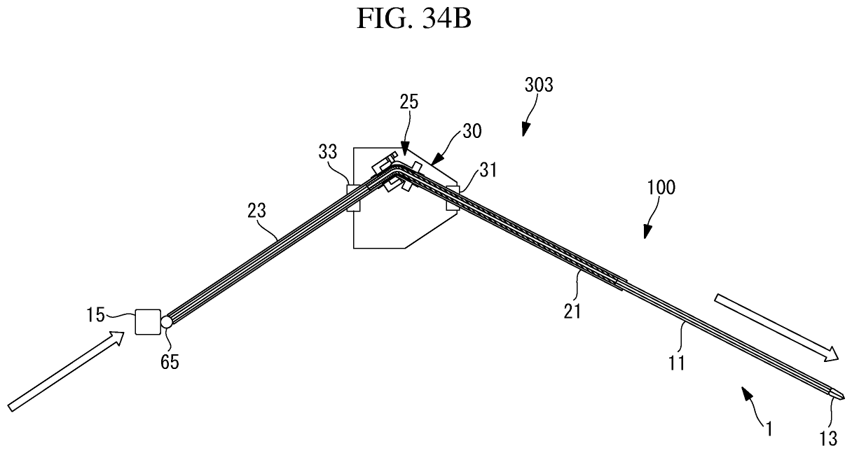

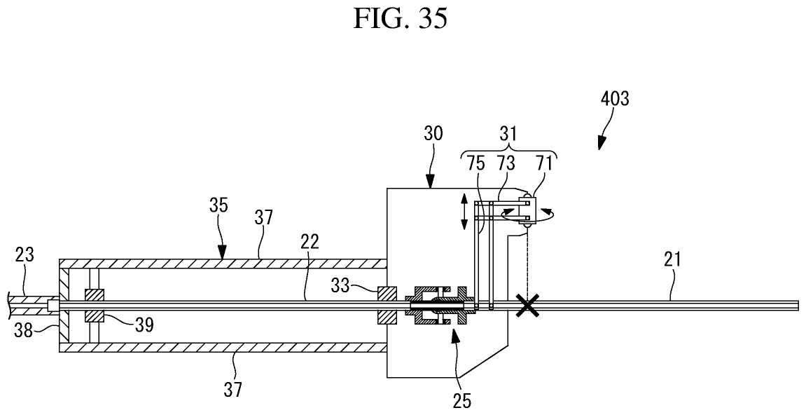

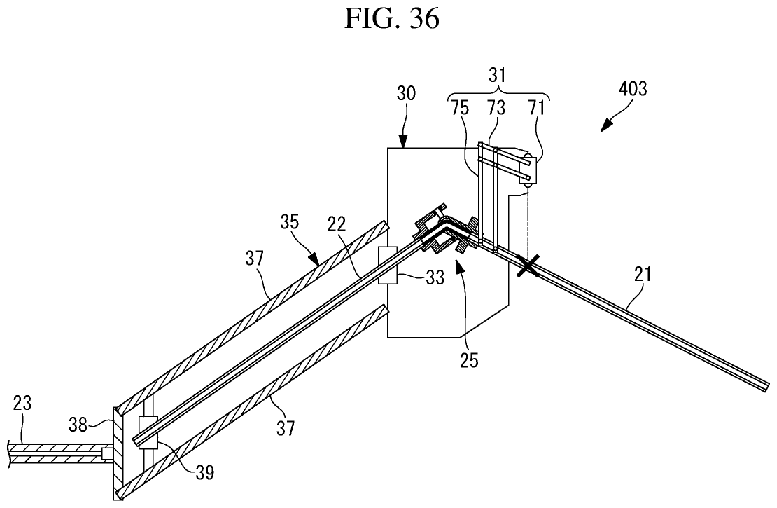

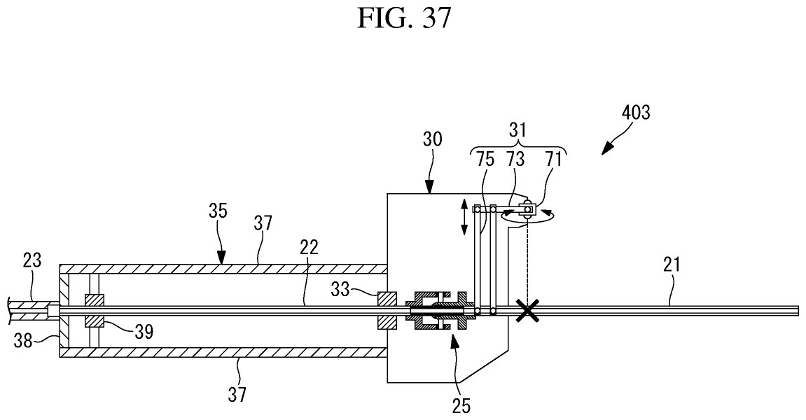

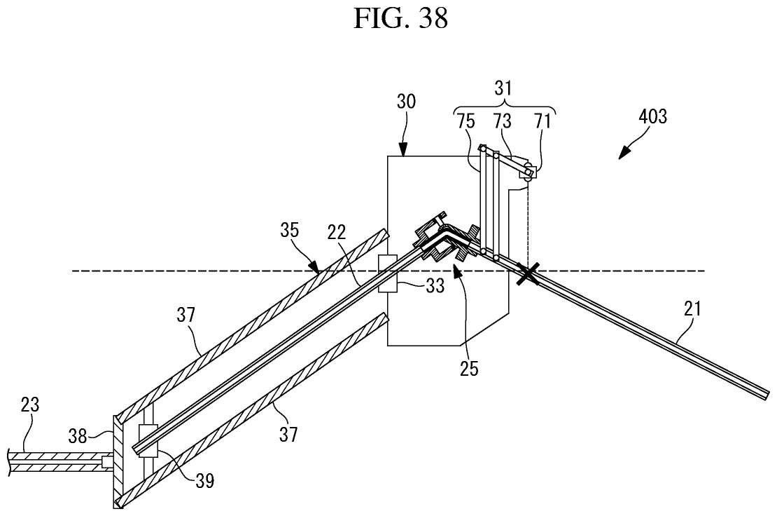

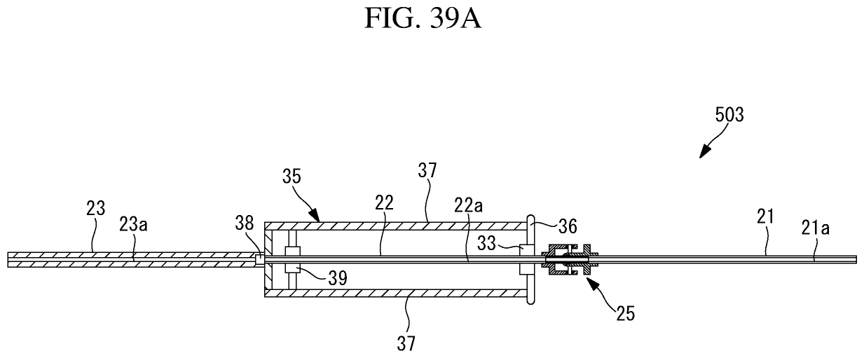

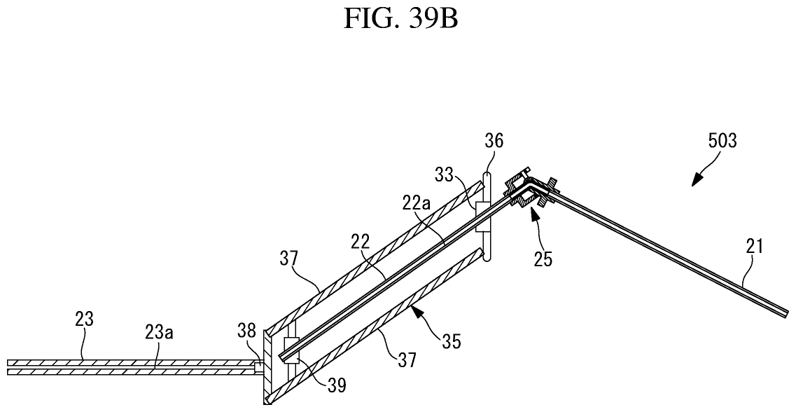

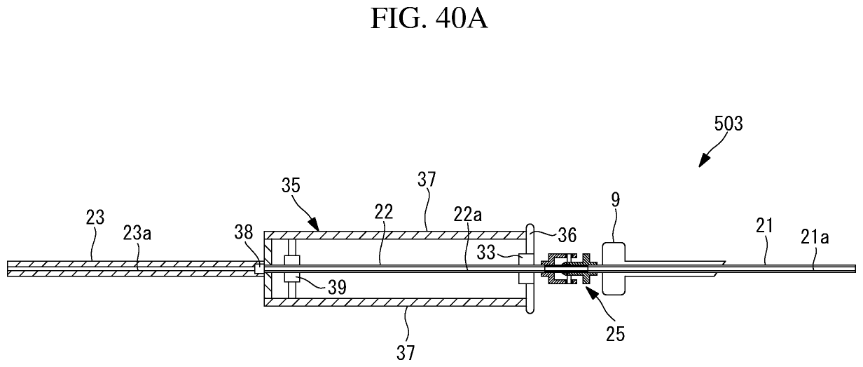

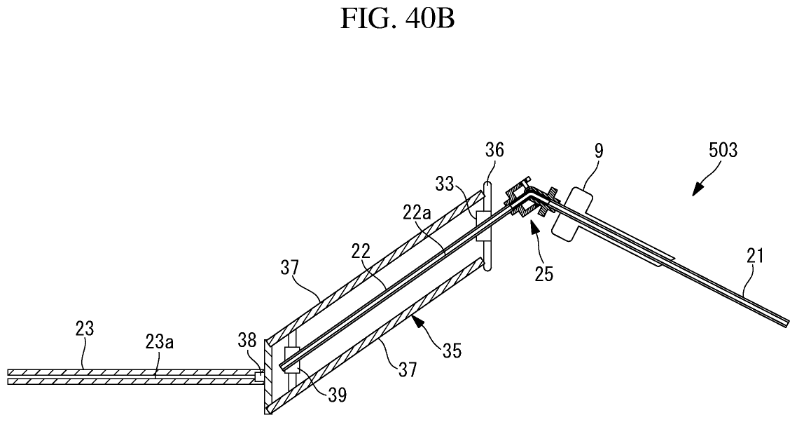

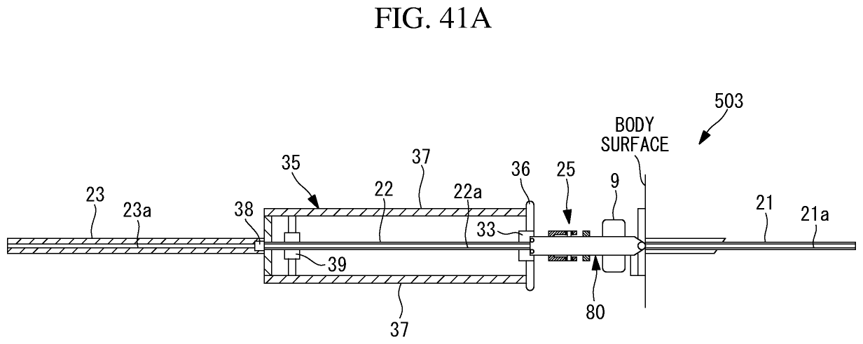

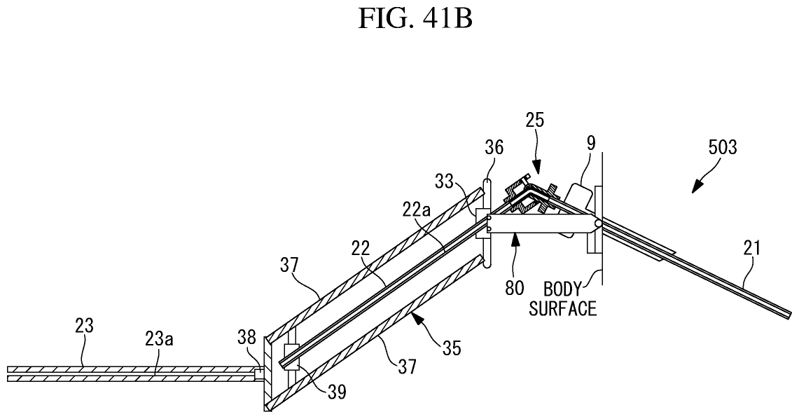

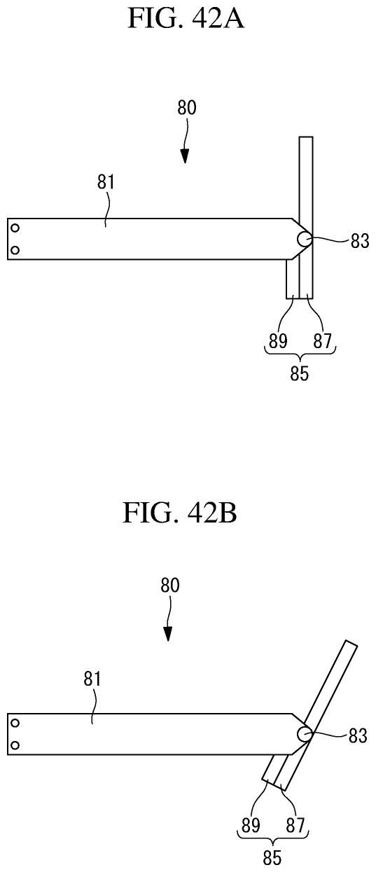

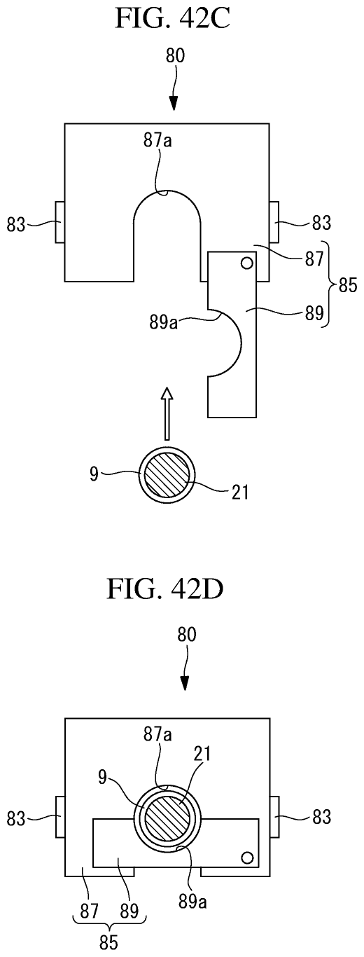

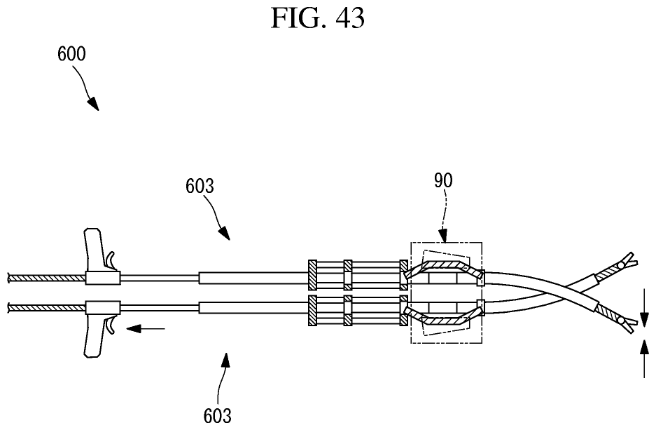

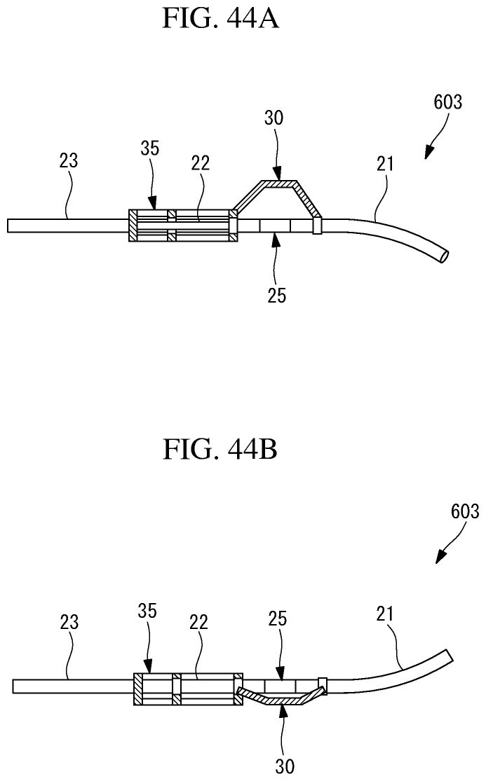

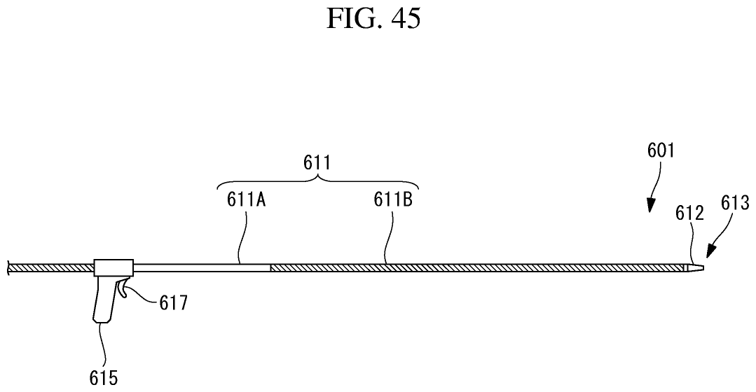

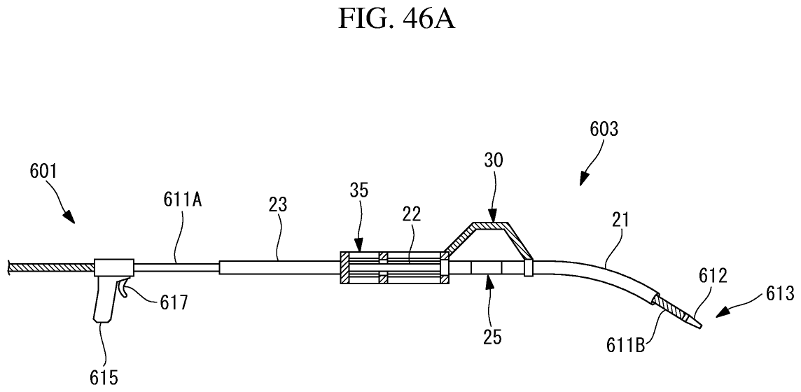

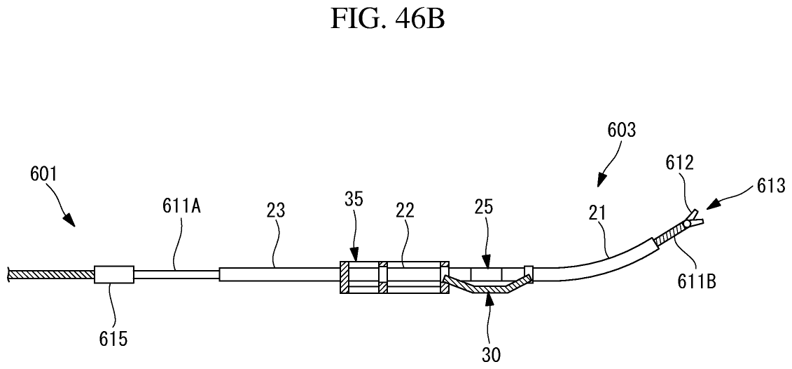

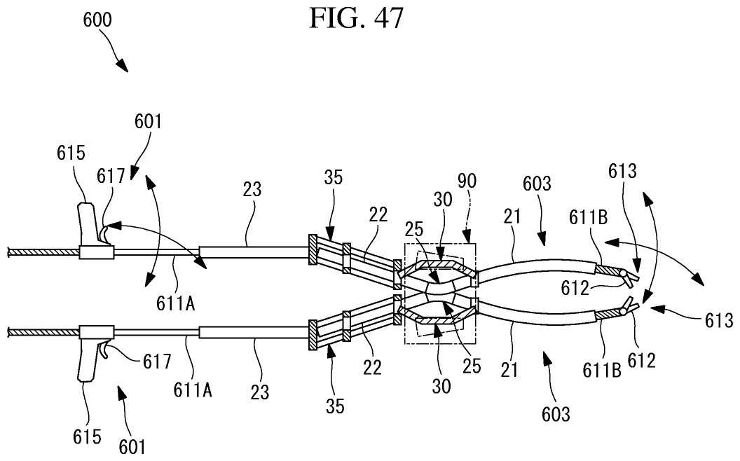

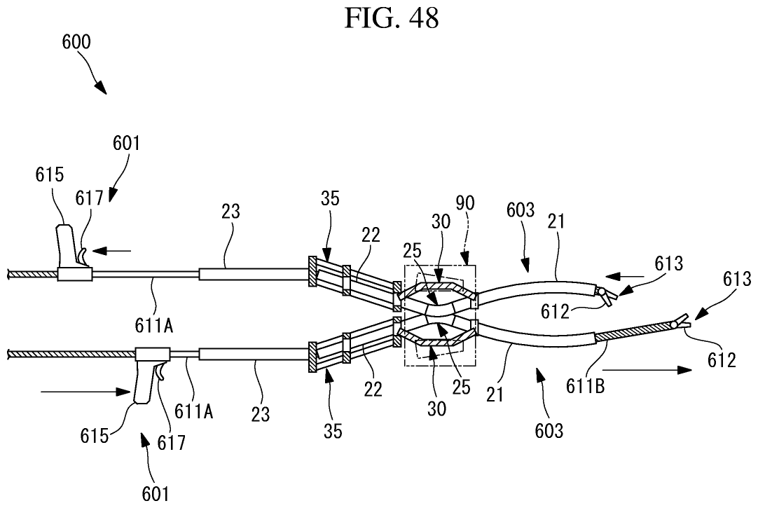

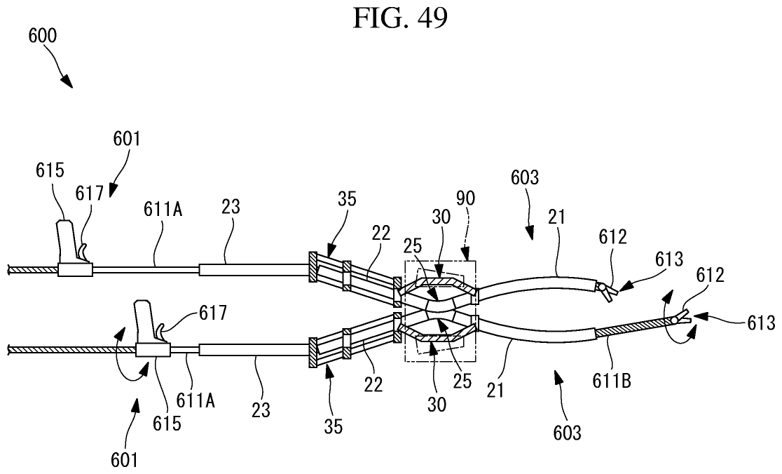

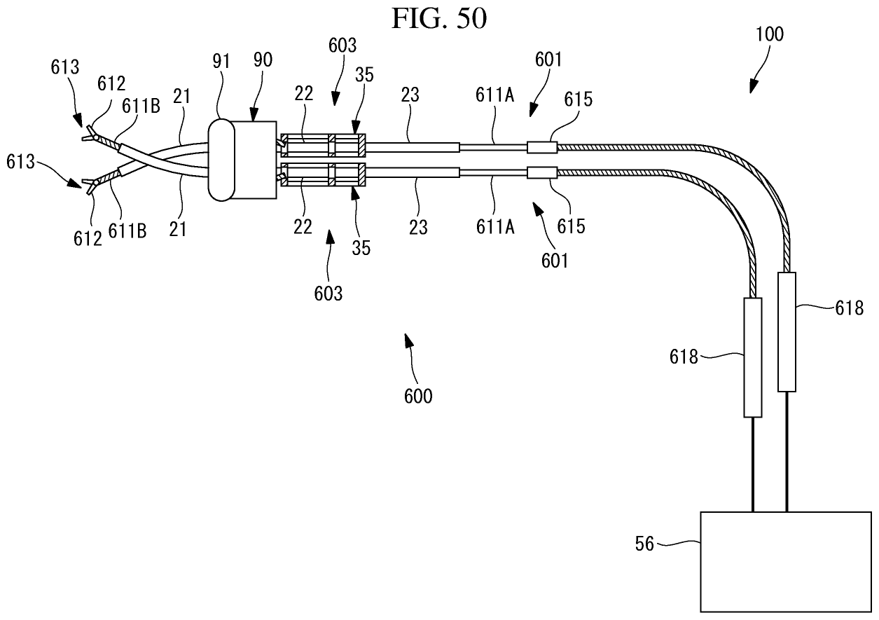

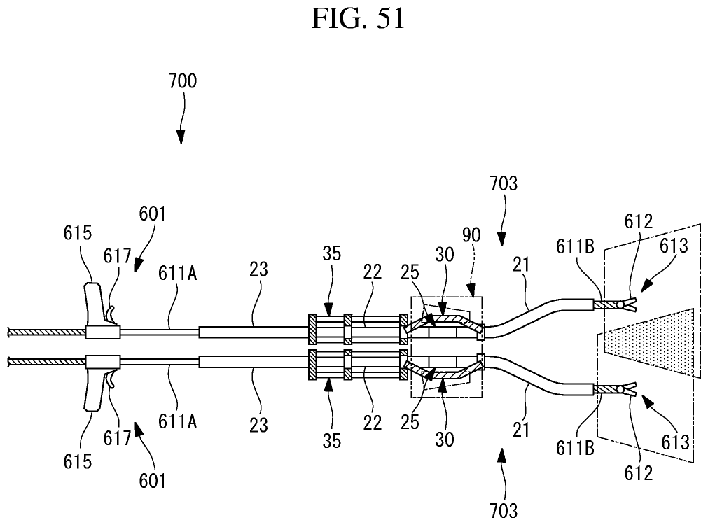

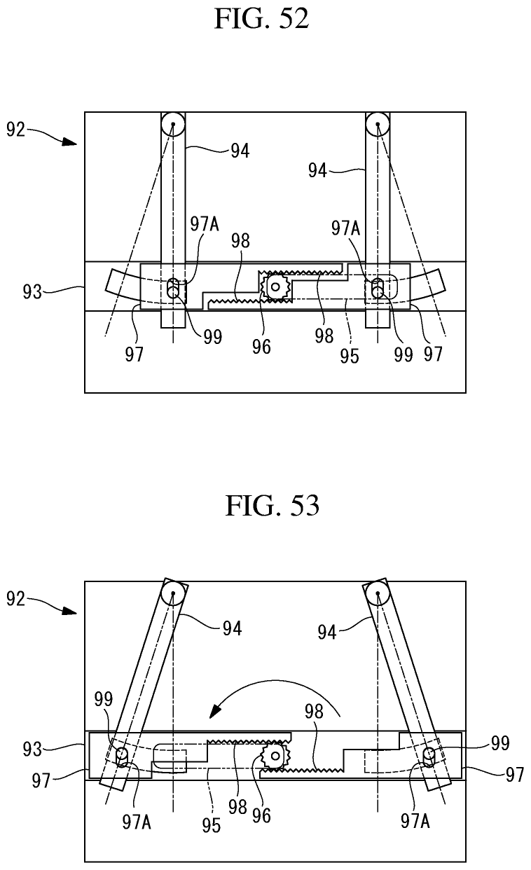

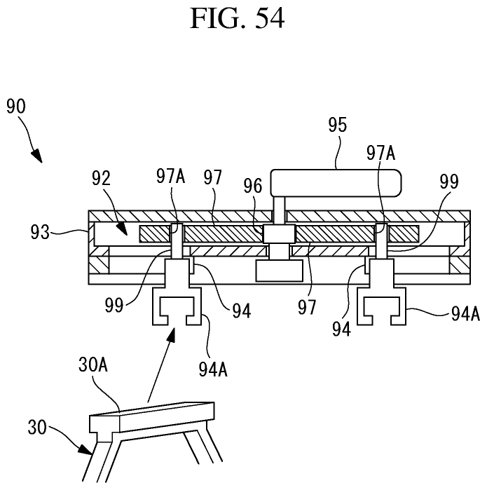

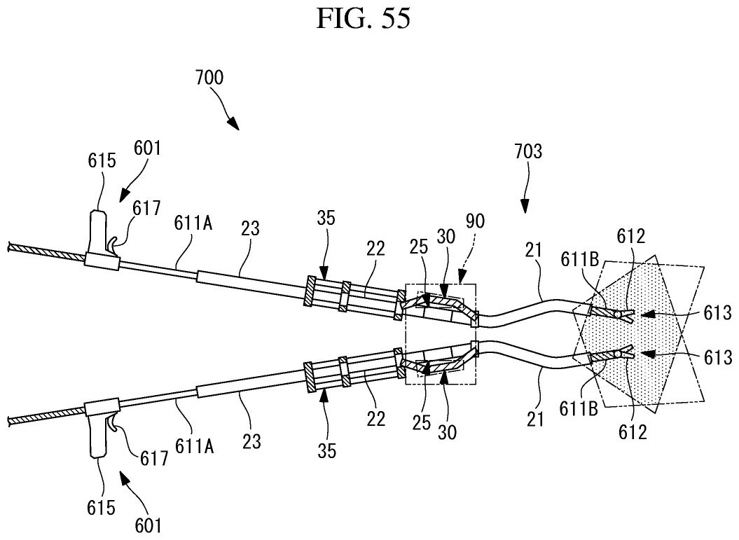

FIG. 10D illustrates a state where the medical treatment device in FIG. 100 is bent together with the cannula.

FIG. 11 FIG. 11 illustrates a state where medical treatment devices are inserted in the body cavity of a patient with two manipulators being disposed parallel to each other.

FIG. 12 FIG. 12 illustrates a state where medical treatment devices are inserted in the body cavity of the patient with the two manipulators being oriented inward.

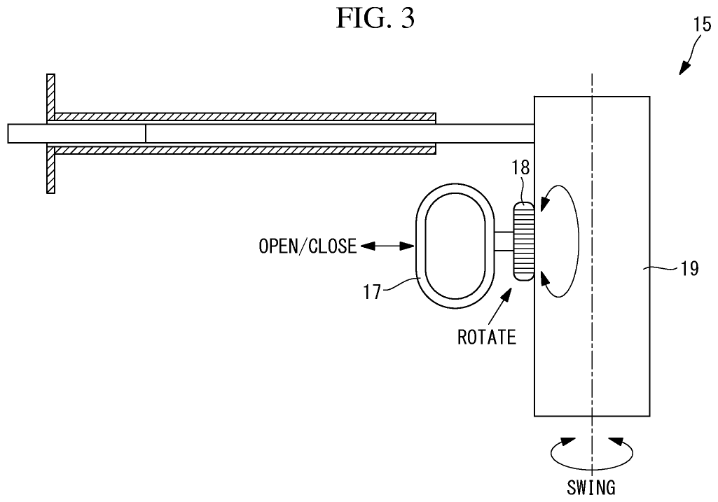

FIG. 13A illustrates an example, as a modification of the handle in FIG. 3, in which an axis of a shaft and an axis of a rotation control section are aligned with each other.

FIG. 13B illustrates an example, as another modification of the handle in FIG. 3, in which the axis of the shaft and the axis of the rotation control section are disposed on different axes that are parallel to each other.

FIG. 13C illustrates an example, as another modification of the handle in FIG. 3, in which the axis of the shaft and the axis of the rotation control section are aligned with each other and in which a rotation axis of a swing control section is disposed in an open-close control section.

FIG. 14 FIG. 14 is a plan view illustrating the connection mechanism having a universal joint structure according to a first modification of the first embodiment of the present invention.

FIG. 15 FIG. 15 is a perspective view illustrating the connection mechanism having a component connection structure according to a second modification of the first embodiment of the present invention.

FIG. 16 FIG. 16 is a vertical sectional view of the front bearing and the rear bearing that use fastening tools according to another modification of the first embodiment of the present invention.

FIG. 17A is a cross-sectional view illustrating the front bearing (or the rear bearing) that supports the first tubular member (or the second tubular member) by means of elastic wires according to a fourth modification of the first embodiment of the present invention.

FIG. 17B illustrates a state where the first tubular member (or the second tubular member) in FIG. 17A is pivoted.

FIG. 18A illustrates the front bearing (or the rear bearing) in FIG. 17A, as viewed in the axial direction.

FIG. 18B illustrates an example, as a modification of the front bearing (or the rear bearing) in FIG. 17A, in which the first tubular member (or the second tubular member) is supported by four elastic wires uniformly arranged in the circumferential direction.

FIG. 18C illustrates an example, as another modification of the front bearing (or the rear bearing) in FIG. 17A, in which the first tubular member (or the second tubular member) is supported by four elastic wires, that is, two on each side, arranged parallel to each other in the radial direction.

FIG. 19A is a plan view illustrating an example where two swing wires are provided in a medical treatment device according to a fifth modification of the first embodiment of the present invention.

FIG. 19B illustrates a state where the gripping section in FIG. 19A is bent.

FIG. 19C is a vertical sectional view of the shaft in FIG. 19A, taken in a direction orthogonal to the longitudinal direction thereof.

FIG. 20A is a plan view illustrating an example where four swing wires are provided in another medical treatment device according to the fifth modification of the first embodiment of the present invention.

FIG. 20B illustrates a state where the gripping section in FIG. 20A is bent.

FIG. 20C is a vertical sectional view of the shaft in FIG. 19A, taken in a direction orthogonal to the longitudinal direction thereof.

FIG. 21A illustrates the overall configuration of a medical treatment device according to a sixth modification of the first embodiment of the present invention.

FIG. 21B illustrates a state where the gripping section in FIG. 21A is bent.

FIG. 22 illustrates the overall configuration of manipulators according to a seventh modification of the first embodiment of the present invention.

FIG. 23A illustrates a reference example of the present invention in which a medical treatment device is manipulated in accordance with a laparoscopic procedure.

FIG. 23B illustrates how the manipulators in FIG. 22 are operated by a surgeon.

FIG. 24 illustrates an example where an affected site in the body cavity of a patient is treated by using the manipulators in FIG. 22.

FIG. 25 illustrates an example where affected sites located at different positions are treated by moving the gripping sections in FIG. 24.

FIG. 26 illustrates a state where two medical treatment devices are inserted into a single port by using the two manipulators in FIG. 22.

FIG. 27 illustrates a detector of a cannula and a manipulator according to a second embodiment of the present invention.

FIG. 28A is a cross-sectional view of a driver of the cannula and the manipulator according to the second embodiment of the present invention.

FIG. 28B is a vertical sectional view of the driver of the cannula and the manipulator according to the second embodiment of the present invention.

FIG. 29A is a cross-sectional view illustrating how a medical treatment device is moved forward by the driver in FIG. 28A.

FIG. 29B is a cross-sectional view illustrating how the medical treatment device is moved rearward by the driver in FIG. 28A.

FIG. 29C is a cross-sectional view illustrating how the medical treatment device is rotated rightward by the driver in FIG. 28A.

FIG. 29D is a cross-sectional view illustrating how the medical treatment device is rotated leftward by the driver in FIG. 28A.

FIG. 30A illustrates an insertion section of a cannula and a manipulator according to a third embodiment of the present invention.

FIG. 30B illustrates a support member and a parallel link of the cannula and the manipulator according to the third embodiment of the present invention.

FIG. 30C illustrates a state where the insertion section in FIG. 30A is inserted in the support member and the parallel link in FIG. 30B.

FIG. 31A illustrates the overall configuration of a cannula according to a fourth embodiment of the present invention.

FIG. 31B illustrates a state where the first tubular member and the second tubular member of the cannula in FIG. 31A are pivoted.

FIG. 32A illustrates the overall configuration of a medical treatment device according to a fourth embodiment of the present invention.

FIG. 32B illustrates the overall configuration of the medical treatment device in FIG. 32A, as viewed from a different angle.

FIG. 33A illustrates a state where the cannula and the medical treatment device according to the fourth embodiment of the present invention are linearly extended.

FIG. 33B illustrates a state where the medical treatment device in FIG. 33A is bent together with the cannula.

FIG. 34A illustrates a state where the medical treatment device is moved forward while the cannula and the medical treatment device in FIG. 33A are linearly extended.

FIG. 34B illustrates a state where the medical treatment device in FIG. 34A is moved forward while being bent.

FIG. 35 is a cross-sectional view of a cannula according to a fifth embodiment of the present invention.

FIG. 36 illustrates a state where a medical treatment device in FIG. 35 is bent in left and right directions.

FIG. 37 is a cross-sectional view illustrating a state where the angle of a rotor in the cannula according to the fifth embodiment of the present invention is changed.

FIG. 38 illustrates a state where the medical treatment device in FIG. 35 is bent in up and down directions.

FIG. 39A is a cross-sectional view of a cannula according to a sixth embodiment of the present invention.

FIG. 39B illustrates a state where the first tubular member and the second tubular member of the cannula in FIG. 39A are pivoted.

FIG. 40A illustrates a state where the first tubular member of the cannula in FIG. 39A is inserted in a trocar.

FIG. 40B illustrates a state where the first tubular member and the second tubular member of the cannula in FIG. 40A are pivoted.

FIG. 41A illustrates a state where the rear bearing of the cannula in FIG. 39A is fixed to the trocar by means of a trocar fastening tool.

FIG. 41B illustrates a state where the first tubular member and the second tubular member of the cannula in FIG. 41A are pivoted.

FIG. 42A is an enlarged view of the trocar fastening tool in FIG. 41A.

FIG. 42B illustrates a state where the angle of an insertion body is changed relative to arms in FIG. 42A.

FIG. 42C illustrates the trocar fastening tool in FIG. 42A, as viewed in the thickness direction of the insertion body.

FIG. 42D illustrates a state where a flip mechanism in FIG. 42C is closed.

FIG. 43 illustrates a manipulator equipped with a cannula system according to a seventh embodiment of the present invention.

FIG. 44A is a front view of a cannula provided in the cannula system in FIG. 43.

FIG. 44B is a side view of the cannula in FIG. 44A.

FIG. 45 is a front view illustrating an example of a medical treatment device provided in the manipulator in FIG. 43.

FIG. 46A is a front view illustrating one of the cannulas and one of the medical treatment devices in the manipulator in FIG. 43.

FIG. 46B is a side view illustrating the cannula and the medical treatment device in FIG. 46A.

FIG. 47 illustrates a state where handles are moved away from each other from the state in FIG. 43.

FIG. 48 illustrates a state where one of the gripping sections is moved forward from the state in FIG. 47.

FIG. 49 illustrates a state where the gripping section moved forward in the state in FIG. 48 is rotated.

FIG. 50 illustrates the overall configuration of the manipulator in FIG. 43.

FIG. 51 illustrates a manipulator equipped with a cannula system according to an eighth embodiment of the present invention.

FIG. 52 illustrates the internal configuration of an angle changing mechanism of a cannula holder provided in the cannula system in FIG. 51.

FIG. 53 illustrates the internal configuration of the cannula holder and shows a state where movable bases are tilted from the state in FIG. 52.

FIG. 54 illustrates a method of fixing each support member to the corresponding movable base of the cannula holder in FIG. 51.

FIG. 55 illustrates a state where the handles are positioned away from each other by actuating the angle changing mechanism from the state in FIG. 51.

### DESCRIPTION OF EMBODIMENTS

### First Embodiment

A cannula 3 and a manipulator 100 according to a first embodiment of the present invention will be described below with reference to the drawings.

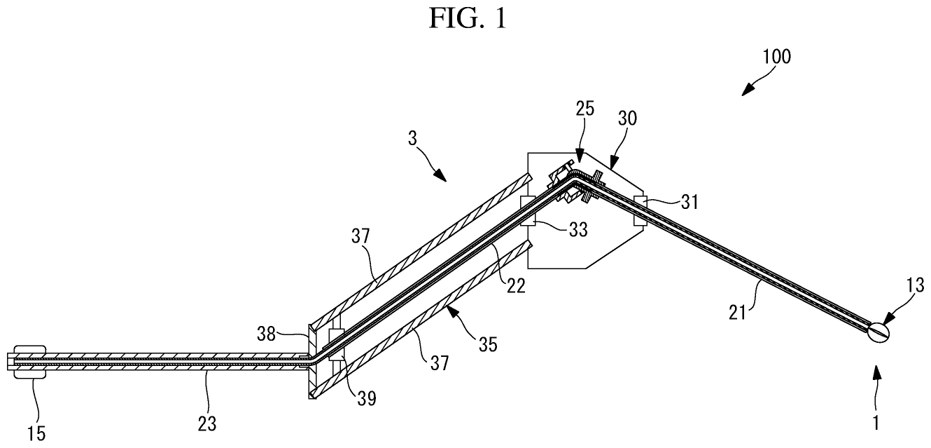

As shown in FIG. 1, the manipulator 100 according to this embodiment includes a medical treatment device 1 used for preforming treatment inside the body cavity of a patient, and also includes the cannula 3 that ensures an insertion path used for inserting the medical treatment device 1 into the body cavity.

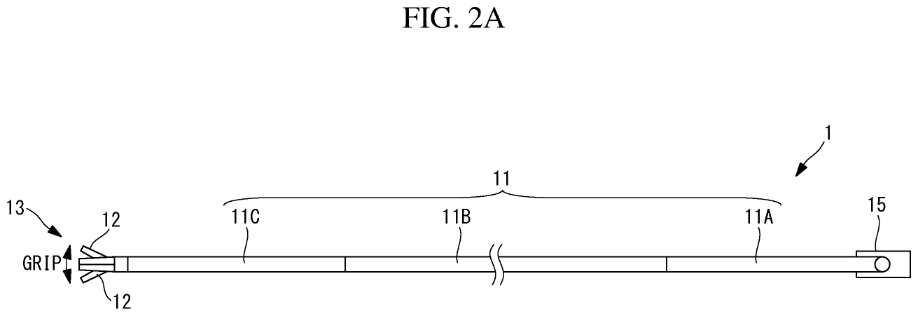

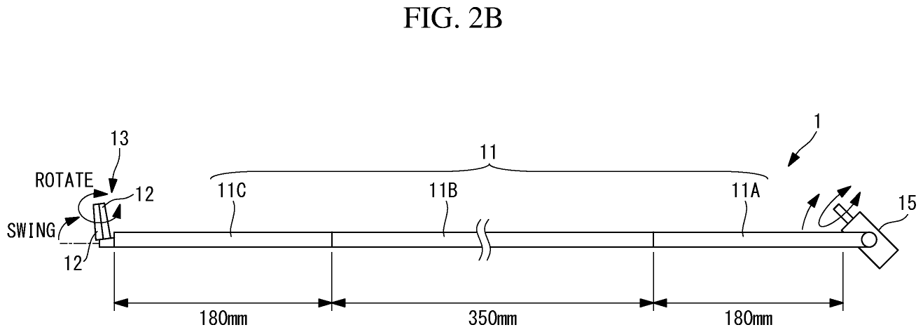

Examples of the medical treatment device 1 include gripping forceps, dissecting forceps, scissors, a high-frequency treatment device, an ultrasonic treatment device, and a needle holder. In this embodiment, gripping forceps shown in FIGS. 2A and 2B will be described as an example of the medical treatment device 1. The medical treatment device 1 includes an elongated shaft 11, a gripping section (distal-end treatment section) 13 connected to the distal end of the shaft 11 and having a pair of openable-closable gripping segments 12, and a handle (proximal-end operating section) 15 that is connected to the proximal end of the shaft 11 and that controls the opening and closing of the gripping segments 12.

In the following order from the proximal end, the shaft 11 is constituted of a first rigid segment 11A formed of a rigid material, such as a pipe, a flexible segment 11B, such as a coil tube, bendable in a direction intersecting the longitudinal direction, and a second rigid segment 11C formed of a pipe similar to the first rigid segment 11A. For example, the shaft 11 has a rigid-flexible-rigid (1:2:1) connection structure from the proximal end, such that the first rigid segment 11A has a length of 180 mm, the flexible segment 11B has a length of 350 mm, and the second rigid segment 11C has a length of 180 mm.

The gripping section 13 includes a link mechanism (not shown) that connects the pair of gripping segments 12 to each other. The pair of gripping segments 12 open and close about the link mechanism acting as an axis. The gripping section 13 is connected to the handle 15 by means of a wire (not shown), such that three degrees of freedom, namely, the opening and closing (i.e., gripping) of the pair of gripping segments 12, the rotation of the shaft 11 around the longitudinal axis, and the swinging of the shaft 11 in the direction intersecting the longitudinal axis, are controllable by using the handle 15.

As shown in FIG. 3, the handle 15 includes an open-close control section 17 for opening and closing the pair of gripping segments 12, a rotation control section 18 for rotating the gripping section 13 around the longitudinal axis of the shaft 11, and a swing control section 19 for swinging the gripping section 13 in the direction intersecting the longitudinal axis of the shaft 11. When the handle 15 is tilted in a yawing direction (i.e., left-right direction), the gripping section 13 is tilted by the same angle as the handle 15.

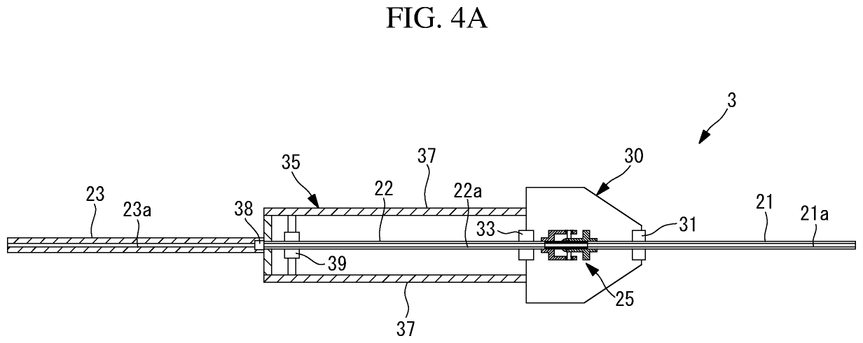

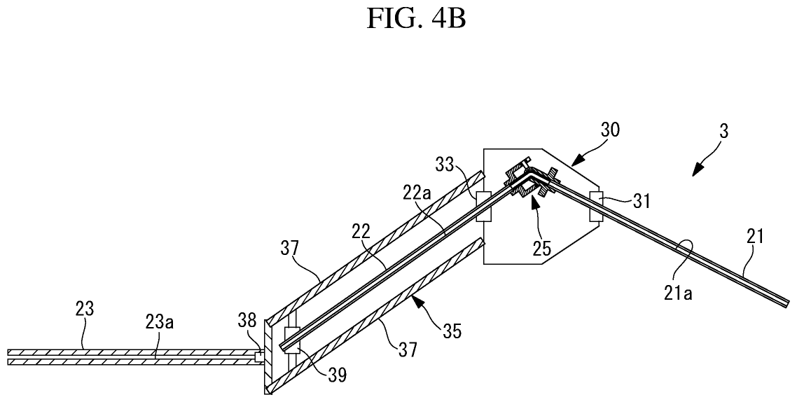

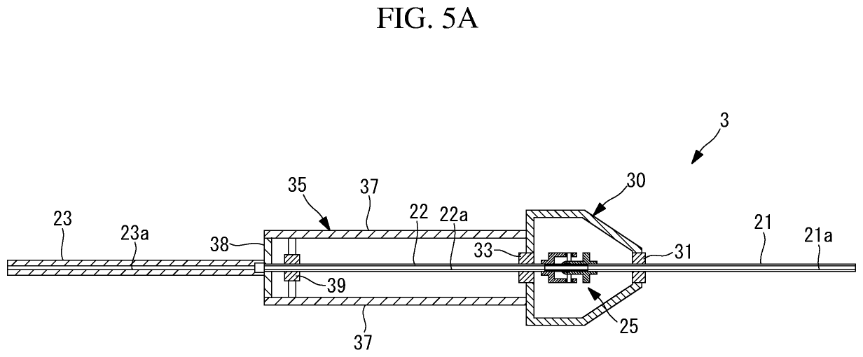

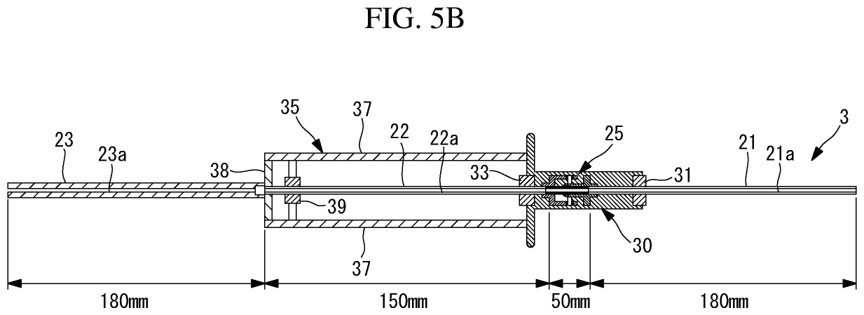

As shown in FIGS. 4A and 4B and FIGS. 5A and 5B, the cannula 3 includes a first tubular member 21, a second tubular member 22, and a third tubular member 23 that are ring-shaped and elongated and are arranged in series, a connection mechanism 25 that connects the first tubular member 21 and the second tubular member 22 to each other, a support member 30 that accommodates the connection mechanism 25 therein and supports the first tubular member 21 and the second tubular member 22 in a state where they extend through the support member 30, and a parallel link 35 connected to the support member 30 and extending parallel to the second tubular member 22.

The first tubular member 21, the second tubular member 22, and the third tubular member 23 respectively have a first through-hole 21a, a second through-hole 22a, and a third through-hole 23a through which the medical treatment device 1 is insertable. The first tubular member 21, the second tubular member 22, and the third tubular member 23, in a linearly extended state, can allow the gripping section 13 of the medical treatment device 1 to continuously move into and out of the through-holes 21a, 22a, and 23a.

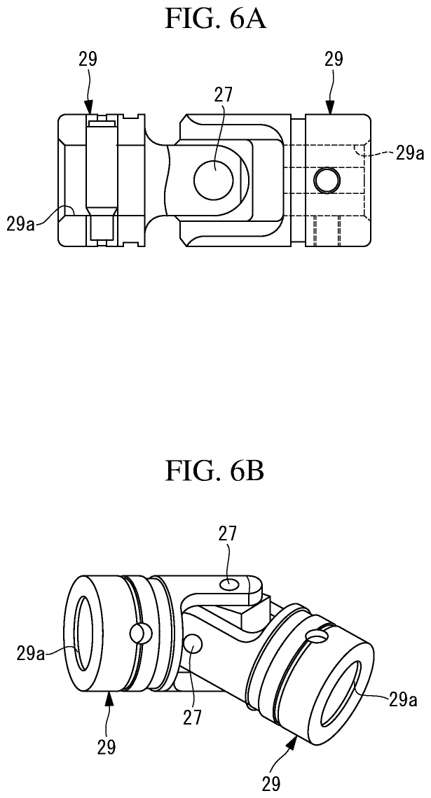

As shown in FIGS. 6A and 6B, the connection mechanism 25 has a universal joint structure obtained by connecting two connection members 29 that are pivotable around rotation shafts 27 extending orthogonally to each other. With the connection mechanism 25, the pivoting of the second tubular member 22 can be transmitted to the first tubular member 21 efficiently with high rigidity. Each of the connection members 29 has a through-hole 29a that engages with the first tubular member 21 or the second tubular member 22.

The connection mechanism 25 arranges the first through-hole 21a of the first tubular member 21 and the second through-hole 22a of the second tubular member 22 in series by means of the two connection members 29 and connects the first tubular member 21 and the second tubular member 22 in a pivotable manner around an axis extending orthogonally to a plane including the axes of the through-holes 21a and 22a.

The support member 30 has a front bearing 31 that supports the first tubular member 21 in a state where the first tubular member 21 extends therethrough, and also has a rear bearing 33 that supports the second tubular member 22 in a state where the second tubular member 22 extends therethrough.

The front bearing 31 and the rear bearing 33 are arranged on the same axis with a distance therebetween, and are secured by means of the support member 30.

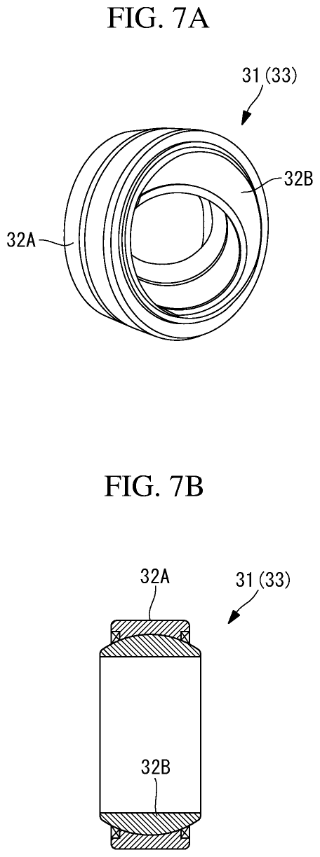

The front bearing 31 and the rear bearing 33 each have a spherical bearing structure shown in FIGS. 7A and 7B. Moreover, the front bearing 31 and the rear bearing 33 each include a ring-shaped outer frame 32A fixed to the support member 30 and a ring-shaped movable section 32B engaged with the outer frame 32A and supported in a movable manner by the outer frame 32A.

The front bearing 31 and the rear bearing 33 respectively allow the first tubular member 21 and the second tubular member 22 to extend through the movable section 32B, and support the first tubular member 21 and the second tubular member 22 in a three-dimensionally pivotable manner (i.e., pivotably movable manner) around an axis intersecting the longitudinal direction.

With each of the front bearing 31 and the rear bearing 33 having such a spherical bearing structure, the first tubular member 21 and the second tubular member 22 pivoted by any angle can be moved with similar low resistance regardless of the pivoting direction and the pivoting angle.

The front bearing 31 supports the first tubular member 21 in a longitudinally positioned state. Therefore, the distal end protruding from the front bearing 31 of the first tubular member 21, that is, an insertion area to be inserted into the body cavity of a patient, constantly has a fixed length (cannula length). In contrast, the rear bearing 33 supports the second tubular member 22 in a slidable manner in the longitudinal direction. Therefore, when the first tubular member 21 and the second tubular member 22 are pivoted by the connection mechanism 25, the second tubular member 22 slides in the longitudinal direction within the rear bearing 33, thereby allowing for smooth movement.

The parallel link 35 includes three or more (four in this embodiment) fulcrum members 37 extending parallel to one another in the longitudinal direction of the second tubular member 22, and also includes a blocking member 38 that blocks the other end of each of the fulcrum members 37.

Each fulcrum member 37 has one end connected to the support member 30 in a three-dimensionally pivotable manner relative to the rear bearing 33 around an axis intersecting the axial direction, and the other end connected in a three-dimensionally pivotable manner relative to the blocking member 38 around an axis intersecting the axial direction. Accordingly, the four fulcrum members 37 can pivot three-dimensionally relative to the support member 30 and the blocking member 38 while maintaining their parallel positions.

The parallel link 35 is provided with a retainer 39 that maintains the four fulcrum members 37 and the second tubular member 22 parallel to one another. The retainer 39 is disposed at the blocking member 38 side of the parallel link 35 in the longitudinal direction and is fixed to the four fulcrum members 37. For example, the retainer 39 is similar to the front bearing 31 and the rear bearing 33 in that it has a spherical bearing structure that allows the second tubular member 22 to extend therethrough and supports the second tubular member 22 in a three-dimensionally pivotable manner around an axis intersecting the longitudinal direction.

The third tubular member 23 has a third through-hole 21c that is disposed in series relative to the second through-hole 22a of the second tubular member 22 and has one end connected to the blocking member 38 of the parallel link 35. With the parallel link 35, the third tubular member 23 and the outer frame 32A of the rear bearing 33 are connected to each other while the axes thereof are maintained in a parallel state.

The operation of the cannula 3 and the manipulator 100 according to this embodiment having the above-described configuration will be described below.

The cannula 3 supports the first tubular member 21 and the second tubular member 22, connected to each other by the connection mechanism 25, in a three-dimensionally pivotable manner around an axis intersecting the longitudinal direction by means of the front bearing 31 and the rear bearing 33 fixed to the support member 30, so that when the first tubular member 21 and the second tubular member 22 are pivoted around the an axis in the connection mechanism 25, the ends of the first tubular member 21 and the second tubular member 22 move in the same direction.

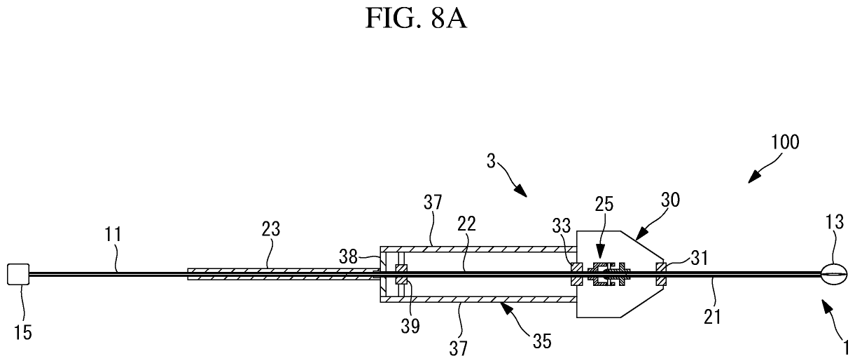

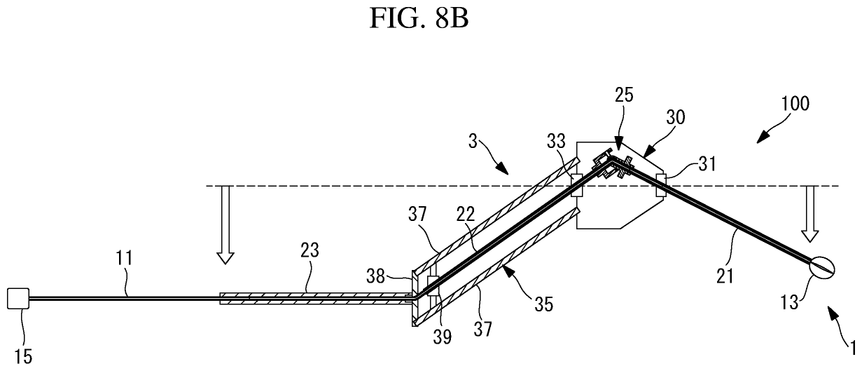

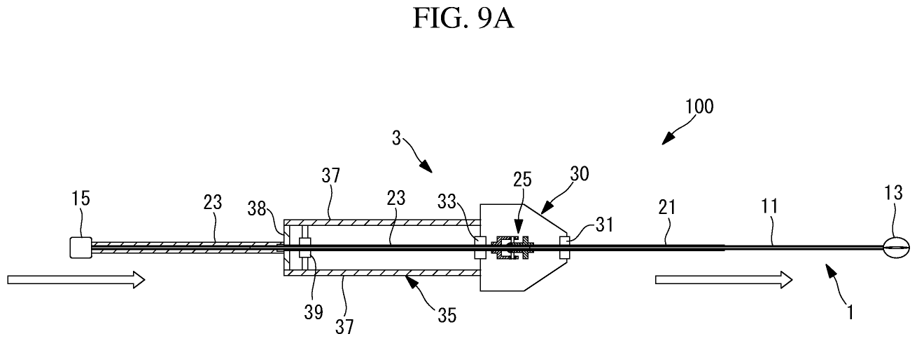

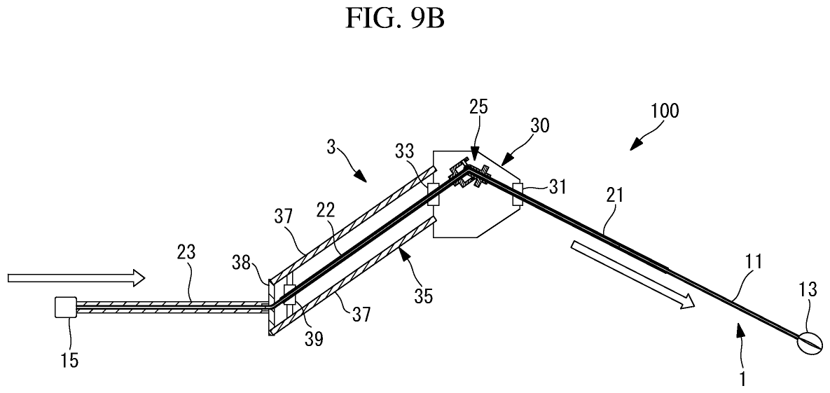

Therefore, as shown in FIGS. 8A and 8B, when the handle 15 is moved in the direction orthogonal to the longitudinal direction of the shaft 11 in a state where the medical treatment device 1 is inserted in the cannula 3, the first tubular member 21 and the second tubular member 22 pivot around the axis and move in the same direction due to the connection mechanism 25, whereby the handle 15 and the gripping section 13 can be moved in the same direction. Furthermore, as shown in FIGS. 9A and 9B, by moving the handle 15 forward, the shaft 11 is moved forward within the cannula 3, so that the gripping section 13 can protrude by a large amount from the distal end of the first tubular member 21.

The following description relates to a case where an affected site within the body cavity of a patient is treated by using the cannula 3 and the manipulator 100 according to this embodiment.

First, based on a procedure similar to that of normal laparoscopic surgery, an observation port (not shown) is attached in a penetrating state to the body wall of the patient, and an endoscope (not shown) is inserted into the body cavity through the port.

Then, the body wall is incised for forming a port installation opening (not shown), and a port 5 is set in the incision, as shown in FIG. 10A. Subsequently, as shown in FIG. 10B, the first tubular member 21 of the cannula 3 is inserted into the port 5 and is supported by the port 5 in a state where the first tubular member 21 extends therethrough, and the cannula 3 is fixed to a surgical table (not shown) by using a surgical arm 7. Alternatively, a cannula holder (not shown) integrated with a surgical arm (not shown) may be set in the port 5 and be fixed to the surgical table, and the cannula 3 may be inserted into and fixed to the cannula holder.

It is desirable that the cannula 3 be inserted into the body cavity in a state where a trocar with a blunt tip is set in the cannula 3 so as not to damage the interior of the body with the tip of a cylinder. Moreover, when the cannula 3 is to be set, it is desirable that the central position of the front bearing 31 be disposed as close to the patient's body surface or the center between the body surface and the peritoneum as possible. Accordingly, the fulcrum to be used when operating the manipulator is located at the body surface or the center between the body surface and the peritoneum, so that the force applied to the patient's body tissue during the operation can be reduced, thereby reducing invasiveness.

Subsequently, a video image of the endoscope inserted in the body cavity is observed to confirm that the distal end of the first tubular member 21 is disposed at a location suitable for the procedure, and the support member 30 of the cannula 3 is fixed to the body wall. After the cannula 3 is fixed, the first tubular member 21, the second tubular member 22, and the third tubular member 23 are linearly extended, as shown in FIG. 100, and the gripping section 13 of the medical treatment device 1 is inserted through the third through-hole 23a in the third tubular member 23. By linearly extending the first tubular member 21, the second tubular member 22, and the third tubular member 23, the gripping section 13, which is rigid, becomes insertable. Accordingly, the gripping section 13 of the medical treatment device 1 is introduced into the body cavity of the patient.

Then, appropriate treatment is performed by using the medical treatment device 1 inserted in the body cavity through the cannula 3. For example, as shown in FIG. 10D, treatment, such as surgery, is performed within the body cavity by operating the handle 15 for guiding the gripping section 13 as well as for controlling, for example, swinging, rotating, and gripping operations. In this case, the manipulator 100 according to this embodiment synchronizes the moving direction of the handle 15 with the moving direction of the gripping section 13, so that the surgeon can perform the operation intuitively.

When the medical treatment device 1 is to be replaced in accordance with the progress of the procedure, the replacing process is performed in a state where the first tubular member 21, the second tubular member 22, and the third tubular member 23 of the cannula 3 are fixed in a linearly extended state, similarly to the inserting process.

As described above, in the cannula 3 and the manipulator 100 according to this embodiment, when the gripping section 13 inserted in the body cavity is to be directly manipulated by using the handle 15 disposed outside the body cavity, the surgeon can perform the operation intuitively by synchronizing the moving direction of the handle 15 with the moving direction of the gripping section 13.

In this embodiment, it is desirable that manipulators 100 be tilted toward the affected site of the patient and the support members 30 be disposed such that the axes of the front bearings 31 are oriented toward the affected site within the body cavity, as shown in FIG. 12, as compared with a case where the manipulators 100 are disposed parallel to each other and the support members 30 are fixed such that the axes of the front bearings 31 are orthogonal to the body wall of the patient, as shown in FIG. 11. Accordingly, the substantial movable range of the gripping sections 13 can be increased. Moreover, an angular deviation occurring when the gripping sections 13 are moved forward and rearward may be reduced, so that the operation can be performed more naturally.

This embodiment relates to an example where two manipulators 100 are used simultaneously. Alternatively, for example, one manipulator 100 may be used alone.

Furthermore, in this embodiment, in order to allow for smooth insertion of the medical treatment device 1 into the cannula 3, the cannula 3 may be provided with a locking mechanism that locks the tubular members 21, 22, and 23 in a linearly extended state. In this case, for example, the locking mechanism may include a movable tubular member (not shown) at the second tubular member 22 side of the third tubular member 23 and may move the movable tubular member to cover the second tubular member 22, thereby achieving the locked state.

Furthermore, in this embodiment, for example, the handle 15 may be configured such that the axis of the shaft 11 and the rotation axis of the rotation control section 18 are aligned with each other, as shown in FIG. 13A. Accordingly, natural movement of the gripping section 13 can be achieved. Alternatively, as shown in FIG. 13B, the axis of the shaft 11 and the rotation axis of the rotation control section 18 may be disposed on different axes that are parallel to each other. As another alternative, as shown in FIG. 13C, the axis of the shaft 11 and the rotation axis of the rotation control section 18 may be aligned with each other, and the rotation axis of the swing control section 19 may be disposed in the open-close control section 17.

This embodiment may be modified as follows.

In this embodiment, the connection mechanism 25 is described as having a universal joint structure obtained by connecting two connection members. As a first modification, for example, a connection mechanism 25A may have a universal joint structure obtained by connecting three or more connection members 29 in series, as shown in FIG. 14.

The connection mechanism 25A shown in FIG. 14 includes three connection members 29. The ends of the connection member 29 in the middle and the connection members 29 at the opposite sides thereof are connected in a pivotable manner around axes intersecting each other. Accordingly, the connected area between the first tubular member 21 and the second tubular member 22 can be pivoted by a large angle while maintaining high rigidity. Consequently, the operating range of the medical treatment device 1 can be increased.

As a second modification, for example, a connection mechanism 25B has a component connection structure obtained by connecting a plurality of components 28, as shown in FIG. 15. Accordingly, the medical treatment device 1 to be inserted into the through-holes 21a and 22a in the first tubular member 21 and the second tubular member 22 can be pivoted by a large fixed angle.

In this modification, the pivoting angle of the connection mechanism 25B can be further increased by increasing the number of components 28. Accordingly, the medical treatment device 1 can be maintained at a large fixed curvature even with a large pivoting angle. This enables smoother forward and rearward movement of the medical treatment device 1, so that the medical treatment device 1 can be moved more finely.

As a third modification, for example, the connection mechanism 25 may have a tube structure like that of a flexible cylindrical tube. Accordingly, the connected area between the first tubular member 21 and the second tubular member 22 can be pivoted by a natural angle due to the connection mechanism 25, thereby allowing for smoother movement of the medical treatment device 1. Consequently, the gripping section 13 can be manipulated more finely.

Furthermore, cost reduction can be achieved, as compared with the case where the connection mechanism 25 has the component connection structure. Therefore, with the connection mechanism 25 being used alone, high cost efficiency can be expected when the connection mechanism 25 is to be replaced each time. The tube structure may be composed of, for example, a simple plastic material, such as PTFE. Moreover, for example, the structure may be increased in strength by covering the plastic material with a metallic mesh.

Furthermore, as an alternative to this embodiment in which the front bearing 31 and the rear bearing 33 each have a spherical bearing structure, at least one of the front bearing 31 and the rear bearing 33 may have a spherical structure. Moreover, for example, as shown in FIG. 16, fastening tools 34 through which the first tubular member 21 (or the second tubular member 22) is to extend may be disposed at opposite sides of the front bearing 31 (or the rear bearing 33) having the spherical structure, and the fastening tools 34 may be screwed to the first tubular member 21 (or the second tubular member 22).

As a fourth modification, as shown in FIGS. 17A and 17B, the front bearing 31 and the rear bearing 33 may have a structure that pulls the first tubular member 21 and the second tubular member 22 from opposite sides evenly in the radial direction by means of elastic wires 41 extending in the radial direction, in place of the spherical structure.

In this case, the first tubular member 21 (or the second tubular member 22) and a ring-shaped frame 43 that surrounds the first tubular member 21 (or the second tubular member 22) may be connected by a plurality of elastic wires 41. It is desirable that the elastic wires 41 be disposed at positions where they are point symmetrical with respect to the center point of the first tubular member 21 (or the second tubular member 22), that is, positions where they are disposed evenly in the circumferential direction of the first tubular member 21 (or the second tubular member 22), so that the force applied to the first tubular member 21 (or the second tubular member 22) is as uniform as possible during the pivoting motion.

For example, if two elastic wires 41 are used, as shown in FIG. 18A, the front bearing 31 (or the rear bearing 33) can have an extremely small area. In particular, reduced invasiveness can be achieved by using two elastic wires 41 in the front bearing 31 that needs to be brought into contact with the patient's body surface.

Alternatively, as shown in FIG. 18B, three or more elastic wires 41 may be disposed uniformly apart from one another in the circumferential direction, so that the resistance acting on the first tubular member 21 (or the second tubular member 22) may be made more uniform during the pivoting motion. As another alternative, as shown in FIG. 18C, in order to achieve both thickness reduction of the front bearing 31 (or the rear bearing 33) and a uniform force applied to the first tubular member 21 (or the second tubular member 22), two elastic wires 41 may be connected to each side of the first tubular member 21 (or the second tubular member 22) in the radial direction, such that the first tubular member 21 (or the second tubular member 22) is pulled from opposite sides by four elastic wires 41.

In this modification, the elastic wires 41 used may be composed of steel. Moreover, if the elastic wires 41 used have a low elastic modulus, the elastic wires 41 may be connected with springs (not shown) for assisting with the expansion and contraction during the pivoting motion. Alternatively, a plastic material, such as polyimide, having both stretchability and strength may be used in place of springs. As another alternative, the first tubular member 21 (or the second tubular member 22) and the frame 43 may be connected by a membrane structure having elasticity, such as a structure composed of rubber, in place of the elastic wires.

As a fifth modification, for example, as shown in FIGS. 19A to 19C, the medical treatment device 1 may include two swing wires 45A and 45B that cause the gripping section 13 to move in a swinging motion, and a single grip-rotation wire 47 that causes the gripping section 13 to move in a rotating motion and a gripping motion. In this case, as shown in FIG. 19C, the handle 15 and the gripping section 13 may be connected by these wires 45A, 45B, and 47.

Furthermore, the swing wires 45A and 45B and the grip-rotation wire 47 may be inserted into coil tubes 49, the grip-rotation wire 47 may be disposed in the middle of the interior of the shaft 11, and the swing wires 45A and 45B may be respectively disposed at the opposite sides of the grip-rotation wire 47 in the radial direction. Accordingly, the two swing wires 45A and 45B can cause the gripping section 13 to bend (yaw or pitch) in two directions intersecting the longitudinal direction.

In this modification, as shown in FIGS. 20A to 20C, four swing wires 45A, 45B, 45C, and 45D may be used. In this case, the four swing wires 45A, 45B, 45C, and 45D may be individually inserted into the coil tubes 49, and may be disposed uniformly apart from one another in the circumferential direction around the grip-rotation wire 47 inside the shaft 11. Accordingly, the four swing wires 45A, 45B, 45C, and 45D can be bent (yawed and pitched) in four directions intersecting the longitudinal direction.

In this modification, the shaft 11 may have a second flexible segment 11B with a component connection structure composed of a flexible material between the second rigid segment 11C and the gripping section 13. Alternatively, the flexible segment 11B may have a component connection structure.

Furthermore, the medical treatment device 1 may be multi-degree-of-freedom forceps in which the gripping section 13 bends and rotates by being driven by a driving force via an elastic wire extending through the shaft 11. By electrically driving the gripping section 13, the operational amount of the handle 15 can be made to match the rotational amount and the bending amount of the gripping section 13 even when the slide resistance of the elastic wire for driving the gripping section 13 changes due to a change in the pivoting angle of the connection mechanism 25. Moreover, the amount of force applied to the handle 15 when being operated can be maintained constant and small, thereby allowing for an accurate and high-quality procedure.

As a sixth modification, for example, as shown in FIGS. 21A and 21B, the shaft 11 may have a second flexible segment 14C, such as a bendable coil tube composed of a flexible material, in place of the second rigid segment 11C. In this case, the second flexible segment 14C may have a bending strength higher than that of the flexible segment 11B. Moreover, the second flexible segment 14C may have a length of 180 mm, similar to the second rigid segment 11C, and the shaft 11 may have a flexible-flexible-rigid (1:2:1) connection structure from the proximal end.

With the distal end of the shaft 11 being bendable due to the second flexible segment 14C in this manner, the forceps are replaceable even in a state where the cannula 3 is bent. In this case, if the distal end of the shaft 11 that is to be exposed to the body cavity from the first tubular member 21 has low elasticity, the distal end of the shaft 11 flexes during an operation, thus making it difficult to achieve the movement desired by the operator. On the other hand, unless the area of the shaft 11 that is bent by the connection mechanism 25 is reduced in elasticity so that the area bends easily, the resistance during the operation increases, thus making it difficult to achieve the movement desired by the operator. By relatively increasing the elasticity of the second flexible segment 14C at the distal end of the shaft 11 that is to be exposed to the body cavity and relatively reducing the elasticity of the flexible segment 11B in the central area that does not protrude from the shaft 11, such conflicting issues can be resolved.

As a seventh modification, as shown in FIG. 22, the rear bearing 33 may be fixable after changing the angle thereof around a pivot axis intersecting the axis of the outer frame 32A. Accordingly, the axis of the outer frame 32A of the rear bearing 33 can be oriented in a direction different from the axis of the outer frame 32A of the front bearing 31.

In this case, one end of each fulcrum member 37 of the parallel link 35 may be directly connected to the rear bearing 33 in a three-dimensionally pivotable manner around an axis orthogonal to the axial direction. Moreover, the rear bearing 33 may be preliminarily changed in angle around the pivot axis before the procedure is performed, and may be fixed to the changed angle during the procedure.

Accordingly, the first tubular member 21 and the second tubular member 22 can be pivoted without being limited by the pivoting angle range of the front bearing 31 or the rear bearing 33. Furthermore, the same angular relationship can be constantly maintained between the rear bearing 33 and the handle 15. Consequently, the angle between the first tubular member 21 that is to be brought into contact with the body surface and the handle 15 can be freely adjusted.

With regard to the positioning of a port in laparoscopic surgery, it is extremely important that the gripping section 13 be set at an appropriate angle (i.e., triangulation) relative to the site to be treated. With this triangulation, the procedure can be carried out appropriately and accurately. On the other hand, since the position of the handle 15 is set in accordance with the position of the port 5, for example, the surgeon may sometimes stretch both arms above the body of the patient to bring the handle 15 to the surgeon's hands, such as when the handle 15 is positioned toward the trunk of the body. In such a case, the surgeon is in a physiologically poor posture and may experience fatigue if the procedure involves long hours, possibly resulting in a low-quality procedure.

As a reference example, for example, in a normal laparoscopic procedure, as shown in FIG. 23A, if the pelvic cavity is to be treated, the surgeon may sometimes need to perform the treatment in a posture that requires an extremely large load by stretching his/her arm straight. In contrast, in this modification, the rear bearing 33 is pivoted around the pivot axis intersecting the axis, so that the surgeon can bend his/her arms and perform the procedure in a comfortable posture, as shown in FIG. 23B.

Furthermore, according to this modification, as shown in FIGS. 24 and 25, by pivoting the rear bearing 33 around the pivot axis intersecting the axis, different positions within the body cavity can be accessed with the gripping section 13 without having to change the position and the angle of the handle 15, that is, without the surgeon changing his/her the posture. Consequently, a wide range within the body cavity can be treated quickly and readily.

Furthermore, according to this modification, the support member 30 may be set at an angle relative to the body wall of the patient, or the rear bearing 33 may be pivoted around the pivot axis intersecting the axis, so that even when a plurality of medical treatment devices 1 are inserted into a single port 5 by using a plurality of manipulators 100, as shown in FIG. 26, the operating space of the surgeon can be ensured, whereby the procedure can be performed appropriately without interference between the handles 15.

### Second Embodiment

Next, a cannula 103 and a manipulator 100 according to a second embodiment of the present invention will be described.

As shown in FIGS. 27 and 28, the cannula 103 and the manipulator 100 according to this embodiment are different from the first embodiment in being provided with a detector 50 that detects the amount of rotation and the amount of forward-rearward movement of the medical treatment device 1 manipulated using the handle 15, and a driver 55 that causes the medical treatment device 1 inserted in the first tubular member 21 to move forward and rearward in the longitudinal direction and to rotate around the axis in accordance with the amount of rotation and the amount of forward-rearward movement detected by the detector 50.

In the following description, sections having configurations identical to those of the cannula 3 and the manipulator 100 according to the first embodiment are given the same reference signs, and descriptions thereof will be omitted.

As shown in FIG. 27, the detector 50 includes a rotational-amount detecting roller 51 that comes into contact with the medical treatment device 1 inserted in the third tubular member 23 so as to be rotated by friction caused as a result of rotation of the medical treatment device 1, a forward-rearward-movement-amount detecting roller 52 that comes into contact with the medical treatment device 1 inserted in the third tubular member 23 so as to be rotated by friction caused as a result of forward or rearward movement of the medical treatment device 1, and detectors 53A and 53B that detect the rotational amounts and the rotational directions of the rotational-amount detecting roller 51 and the forward-rearward-movement-amount detecting roller 52.

The rotational-amount detecting roller 51 is rotatable around an axis parallel to the axis of the third tubular member 23.

The forward-rearward-movement-amount detecting roller 52 is rotatable around an axis orthogonal to the axis of the third tubular member 23.

The detectors 53A and 53B transmit the detected rotational amounts and the detected rotational directions of the rotational-amount detecting roller 51 and the forward-rearward-movement-amount detecting roller 52 to the driver 55.

As shown in FIGS. 28A and 28B, the driver 55 includes motors 57A and 57B that are accommodated in the support member 30 and that rotate around predetermined rotation axes intersecting the longitudinal direction of the first tubular member 21 and parallel to each other, rollers 59A and 59B connected to the motors 57A and 57B and rotated around the same axes as the motors 57A and 57B, and a controller 56 that drives the motors 57A and 57B in accordance with the rotational amounts and the rotational directions of the rotational-amount detecting roller 51 and the forward-rearward-movement-amount detecting roller 52 transmitted from the detectors 53A and 53B.

The motor 57A and the roller 59A, as well as the motor 57B and the roller 59B, are provided between the front bearing 31 and the connection mechanism 25 and are disposed with the first tubular member 21 interposed therebetween in the radial direction.

The motor 57A and the motor 57B are capable of rotating independently of each other.

The roller 59A and the roller 59B are disposed at an angle of 45 degrees relative to the first tubular member 21 and are brought into contact with the shaft 11 of the medical treatment device 1. Furthermore, the roller 59A and the roller 59B rotate outward to cause the shaft 11 to move forward, as shown in FIG. 29A, and rotate inward to cause the shaft 11 to move rearward, as shown in FIG. 29B. Moreover, the roller 59A and the roller 59B rotate counterclockwise with respect to the advancing direction to cause the shaft 11 to rotate rightward with respect to the advancing direction, as shown in FIG. 29C, and rotate clockwise with respect to the advancing direction to cause the shaft 11 to rotate leftward with respect to the advancing direction, as shown in FIG. 29D.

Based on the amount of rotation and the amount of forward-rearward movement transmitted from the detectors 53A and 53B of the detector 50, the controller 56 causes the roller 59A and the roller 59B to rotate in rotational directions corresponding to the rotational directions of the rotational-amount detecting roller 51 and the forward-rearward-movement-amount detecting roller 52, and causes the roller 59A and the roller 59B to rotate by rotational amounts equal to the rotational amounts of the rotational-amount detecting roller 51 and the forward-rearward-movement-amount detecting roller 52.

In the cannula 103 and the manipulator 100 according to this embodiment having the above-described configuration, the detector 50 and the driver 55 can cause the gripping section 13 of the medical treatment device 1 to actually rotate and move forward and rearward by an amount of rotation and an amount of forward-rearward movement that match the amount of rotation and the amount of forward-rearward movement of the medical treatment device 1 manipulated by using the handle 15.

Accordingly, even when the slide resistance of the medical treatment device 1 changes due to a change in the pivoting angle of the connection mechanism 25, the operational amount of the handle 15 can be made to match the amount of movement of the gripping section 13, and the amount of force applied to the handle 15 when being operated can be maintained constant and small, thereby allowing for an accurate and high-quality procedure.

### Third Embodiment

Next, a cannula 203 and a manipulator 100 according to a third embodiment of the present invention will be described.

As shown in FIGS. 30A to 30C, the cannula 203 and the manipulator 100 according to this embodiment are different from the first embodiment in being provided with an insertion section 60 having the first tubular member 21, the second tubular member 22, and the third tubular member 23 as a single unit, and in that the insertion section 60, the support member 30, and the parallel link 35 have isolated structures.

In the following description, sections having configurations identical to those of the cannulas 3 and 103 and the manipulator 100 according to the first and second embodiments are given the same reference signs, and descriptions thereof will be omitted.

In the insertion section 60, an area corresponding to the first tubular member 21 is composed of a rigid material, and areas corresponding to the second tubular member 22 and the third tubular member 23 are composed of a flexible material.

The parallel link 35 is provided with a tubular member 61 into which the insertion section 60 is insertable, and the insertion section 60 can be secured by being inserted into the tubular member 61.

In the cannula 203 according to this embodiment having the above-described configuration, the insertion section 60 alone can be removed so that the insertion section 60 can be cleaned and sterilized. Moreover, the support member 30 and the parallel link 35 may be used continuously, whereas the insertion section 60 may be replaced after each use.

### Fourth Embodiment

Next, a cannula 303 and a manipulator 100 according to a fourth embodiment of the present invention will be described.

As shown in FIGS. 31A and 31B, the cannula 303 and the manipulator 100 according to this embodiment are different from the first to third embodiments in being not provided with the parallel link 35 and the third tubular member 23.

In the following description, sections having configurations identical to those of the cannulas 3, 103, and 203 and the manipulator 100 according to the first to third embodiments are given the same reference signs, and descriptions thereof will be omitted.

As shown in FIGS. 32A and 32B, in the medical treatment device 1, the first rigid segment 11A, the flexible segment 11B, and the second rigid segment 11C have the same length, and are slightly shorter than the first tubular member 21. The handle 15 and the second rigid segment 11C are connected by a joint member 65 pivotable in up-down and left-right directions. With the joint member 65, the handle 15 can be maintained at a desired angle, regardless of the angle of the second rigid segment 11C.

The operation of the cannula 303 and the manipulator 100 having the above-described configuration will now be described.

With regard to the cannula 303 and the manipulator 100 according to this embodiment, when the handle 15 is moved in a direction intersecting the longitudinal direction of the shaft 11 in a state where the medical treatment device 1 is inserted in the cannula 303, as shown in FIGS. 33A and 33B, the gripping section 13 can be moved in the moving direction of the handle 15. Furthermore, when the handle 15 is moved forward, the gripping section 13 can be moved forward, as shown in FIGS. 34A and 34B.

As described above, in the cannula 303 and the manipulator 100 according to this embodiment, the pivotable joint member 65 allows the handle 15 to be maintained at a fixed angle even when the angle of the second rigid segment 11C is changed. Therefore, the moving direction of the handle 15 can be synchronized with the moving direction of the gripping section 13, so that the surgeon can perform the operation intuitively.

### Fifth Embodiment

Next, a cannula 403 and a manipulator 100 according to a fifth embodiment of the present invention will be described.

As shown in FIGS. 35 and 36, the cannula 403 and the manipulator 100 according to this embodiment are different from the first to fourth embodiments in that the front bearing 31 is offset from the axis of the first tubular member 21.

In the following description, sections having configurations identical to those of the cannulas 3, 103, 203, and 303 and the manipulator 100 according to the first to fourth embodiments are given the same reference signs, and descriptions thereof will be omitted.

In the cannula 403, the front bearing 31 includes a rotor 71 disposed away from the first tubular member 21 and rotatable around a rotation axis intersecting the axis of the rear bearing 33, a pair of first links 73 each having one end connected to the rotor 71 and extending parallel to the first tubular member 21 and to each other, and a pair of second links 75 extending parallel to each other and each having one end connected to the corresponding first link 73 and the other end connected to the first tubular member 21.

The rotor 71 and the first links 73, the first links 73 and the second links 75, and the second links 75 and the first tubular member 21 are connected to each other in a pivotable manner around a pivot axis orthogonal to the rotation axis of the rotor 71.

The operation of the cannula 403 having the above-described configuration will now be described.

As shown in FIGS. 35 and 36, the first links 73 and the second links 75 are caused to pivot around the pivot axis, so that the first tubular member 21 can be pivoted in the same plane as the first links 73 and the second links 75. Moreover, by rotating the rotor 71 around the rotation axis, the first tubular member 21 can be pivoted in a plane intersecting the first links 73 and the second links 75.

In this case, the front bearing 31 is connected to the first tubular member 21 by means of the second links 75, so that the first tubular member 21 can be supported in a three-dimensionally pivotable manner without having to dispose a bearing structure on the axis of the first tubular member 21. Accordingly, the first tubular member 21 can be inserted into the body cavity through a normal trocar to be attached to the body wall of the patient. Moreover, a pivot point (indicated with an X in the drawing) where the first tubular member 21 pivots three-dimensionally can be disposed at the center of the body wall of the patient in the thickness direction, so that a procedure with reduced invasiveness can be achieved.

As an alternative to this embodiment in which the first links 73 are a pair of shafts that are parallel to each other, a first link 73 constituted of a single shaft is also possible, as shown in FIGS. 37 and 38. Even in such a case, advantages similar to those in the case where the first links 73 are constituted of two parallel shafts are achieved.

### Sixth Embodiment

Next, a cannula 503 and a manipulator 100 according to a sixth embodiment of the present invention will be described.

As shown in FIGS. 39A and 39B, the cannula 503 according to this embodiment is different from the first to fifth embodiments in that the support member 30 and the front bearing 31 are not provided, and that the first tubular member 21 is inserted in a trocar 9 attached to the body wall of the patient and is supported the trocar 9 in a state where the first tubular member 21 extends therethrough, as shown in FIGS. 40A and 40B.

In the following description, sections having configurations identical to those of the cannulas 3, 103, 203, 303, and 403 and the manipulator 100 according to the first to fifth embodiments are given the same reference signs, and descriptions thereof will be omitted.

In this embodiment, as shown in FIGS. 41A and 41B, the parallel link 35 includes a blocking member 36 that blocks one end of each fulcrum member 37 and secures the rear bearing 33, and also includes a trocar fastening tool 80 that fixes the rear bearing 33 to the body wall of the patient via the trocar 9.

As shown in FIGS. 41A, 41B, 42A, and 42B, the trocar fastening tool 80 includes a pair of arms 81 each having one end fixed to the parallel link 35, and a trocar insertion section 85 that is connected to the other end of each of the pair of arms 81, secures the trocar 9 by having the trocar 9 extending therethrough, and is to be attached to the body surface of the patient. In the drawings, only one of the arms 81 is shown.

The pair of arms 81 extend in the longitudinal direction of the cannula 3 and are disposed parallel to each other with the connection mechanism 25 interposed therebetween. Each arm 81 has one end connected to the blocking member 36 of the parallel link 35 by means of a screw, and the other end attached to the trocar insertion section 85 in a pivotable manner by means of a hinge 83.

As shown in FIGS. 42C and 42D, the trocar insertion section 85 includes a plate-shaped insertion body 87 having a recessed cutout 87a into which the trocar 9 is fittable, and also includes a flip mechanism 89 that partially blocks the cutout 87a in the insertion body 87 and secures the trocar 9 in the cutout 87a in a state where the trocar 9 extends therethrough.

The insertion body 87 is attached to the other end of each of the pair of arms 81 by means of the hinge 83 and can change the angle relative to the arms 81 by means of the hinge 83. The insertion body 87 is attached to the body surface of the patient so as to support the parallel link 35 via the arms 81.

The cutout 87a extends from an edge of the insertion body 87 to near the center thereof, and has a width that is slightly larger than the diameter of the trocar 9. Moreover, the terminal end of the cutout 87a has a substantially circular-arc shape that conforms to the outer peripheral shape of the trocar 9.

The flip mechanism 89 is capable of blocking the opening of the cutout 87a. The flip mechanism 89 has a recess 89a with a substantially circular-arc shape that conforms to the outer peripheral shape of the trocar 9. The flip mechanism 89 blocks the opening of the cutout 87a so that the recess 89a limits the cutout 87a to a through-hole through which the trocar 9 is extendable. Specifically, when the flip mechanism 89 opens, the trocar 9 can be fitted into the cutout 87a. When the flip mechanism 89 closes in a state where the trocar 9 is fitted in the cutout 87a, the trocar 9 can be fixed in the cutout 87a in a state where the trocar 9 extends therethrough.

It is more preferable that the cutout 87a be large enough to have a gap with respect to the trocar 9 when the trocar 9 is fitted in the cutout 87a and the flip mechanism 89 is closed. By having a gap between the cutout 87a and the trocar 9, the degree of freedom for the pivoting motion of the trocar 9 can be ensured, thereby achieving improved operability.

The operation of the cannula 503 and the manipulator 100 having the above-described configuration will now be described.

When an affected site within the body cavity of a patient is to be treated by using the cannula 503 and the manipulator 100 according to this embodiment, the first tubular member 21 of the cannula 3 is first inserted into the trocar 9 attached to the body wall of the patient. Then, the trocar 9 is fitted into the cutout 87a in the insertion body 87 of the trocar fastening tool 80, the flip mechanism 89 is closed, the insertion body 87 is set on the body surface of the patient, and the cannula 3 is fixed by using, for example, a surgical arm (not shown).

Accordingly, even if the cannula 503 is not provided with the front bearing 31, a fixed relative position of the pivot point where the first tubular member 21 pivots three-dimensionally can always be maintained. Therefore, this configuration is similar to each of the above-described embodiments in that the moving direction of the handle 15 can be synchronized with the moving direction of the gripping section 13, so that the surgeon can manipulate the medical treatment device 1 intuitively by using the trocar 9 as a fulcrum.

As an alternative to this embodiment in which the trocar fastening tool 80 is provided, it is possible to operate the cannula 503 without the trocar fastening tool 80. If the trocar fastening tool 80 is not provided, for example, the distance between the body surface and the blocking member 36 may be measured and adjusted such that the distance is fixed when the cannula 503 is set on the body surface of the patient.

Although the embodiments of the present invention have been described above in detail with reference to the drawings, specific configurations are not limited to these embodiments, and design alterations are also included so long as they do not depart from the scope of the invention. For example, the present invention is not limited to each of the above-described embodiments and modifications and may be applied to an embodiment obtained by appropriately combining these embodiments and modifications; it is not particularly limited.

Furthermore, as an alternative to each of the above-described embodiments in which gripping forceps are described as an example of the medical treatment device 1, other examples include dissecting forceps, scissors, a high-frequency treatment device, an ultrasonic treatment device, a needle holder, a treatment tool used in laparoscopic surgery, such as a clip for sealing a blood vessel, a stapler for cutting and sealing tissue, a basket for collecting tissue, or a water suction tube, and an observation device, such as an endoscope.

Next, a cannula system 600 according to a seventh embodiment of the present invention will be described below with reference to the drawings.

As shown in FIG. 43, the cannula system 600 according to this embodiment includes two cannulas 603 and a cannula holder 90 that supports the cannulas 603.

As shown in FIGS. 44A and 44B, each cannula 603 is similar to the cannula 3 in FIG. 4A in that it includes a first tubular member 21, a second tubular member 22, and a third tubular member 23 that are ring-shaped and elongated and are arranged in series, a connection mechanism 25 that connects the first tubular member 21 and the second tubular member 22 to each other, a support member 30 that is located at the opposite sides of the connection mechanism 25 and that supports the first tubular member 21 and the second tubular member 22 in a state where they extend through the support member 30, and a parallel link 35 that is connected to the support member 30 and extends parallel to the second tubular member 22.

This embodiment is different from the cannula 3 according to the first embodiment in that the first tubular member 21 is bent in one direction.

Furthermore, as shown in FIG. 45, a medical treatment device 601 used together with the cannula system 600 according to this embodiment includes an elongated shaft 611, a gripping section (distal-end treatment section) 613 connected to the distal end of the shaft 611 and having a pair of openable-closable gripping segments 612, and a handle (proximal-end operating section) 615 that is connected to the proximal end of the shaft 611 and that controls the opening and closing of the gripping segments 612.

In the following order from the proximal end, the shaft 611 is constituted of a rigid segment 611A formed of a rigid material, such as a pipe, and a semi-rigid segment 611B formed of a plastic pipe that is bendable in a direction intersecting the longitudinal direction. As shown in FIGS. 46A and 46B, the semi-rigid segment 611B can be freely bent at the connection mechanism 25, and bends in conformity to the shape of the first tubular member 21 within the bent first tubular member 21.

As shown in FIG. 43, in a state where the third tubular members 23 of the two cannulas 603 are disposed close to each other, the cannula holder 90 secures the support members 30 of the two cannulas 603 to achieve a positional relationship where the first tubular member 21 of one of the cannulas 603 bends toward the other cannula 603 such that the two first tubular members 21 intersect each other.

The operation of the cannula system 600 according to this embodiment will be described below.

When the medical treatment devices 601 are inserted into the two cannulas 603 of the cannula system 600 according to this embodiment, the bent first tubular members 21 intersect each other in a state where the third tubular members 23 are disposed closest to each other, as shown in FIG. 43, such that the two gripping sections 613 are disposed at different positions. From this state, the third tubular members 23 are translationally moved away from each other, as shown in FIG. 47, so that the intersecting state between the first tubular members 21 becomes canceled, whereby the two gripping sections 613 can be disposed close to each other.

Specifically, in the cannula system 600 according to this embodiment, the two gripping sections 613 are brought close to each other in a state where the handles 615 of the two medical treatment devices 601 are positioned away from each other, so that when the same target site is to be treated by using the two medical treatment devices 601, interference between the handles 615 can be prevented. This is advantageous in that reduced workability for the surgeon can be prevented.

In this cannula system 600, an open-close control section 617 of each handle 615 may be operated to open and close the pair of gripping segments 612 of each gripping section 613, as shown in FIG. 43, the handle 615 may be moved in the longitudinal direction of the third tubular member 23 to move the gripping section 613 forward and rearward, as shown in FIG. 48, the handle 615 may be moved in the direction intersecting the longitudinal axis of the third tubular member 23 to move the gripping section 613 in the same direction as the handle 615, as shown in FIG. 47, and the handle 615 may be rotated around the longitudinal axis of the third tubular member 23 to rotate the gripping section 613 around the longitudinal axis of the semi-rigid segment 611B, as shown in FIG. 49. Furthermore, the handle 615 may be tilted in the yawing direction (i.e., left-right direction) to tilt the gripping section 613 by the same angle as the handle 615.

Furthermore, as shown in FIG. 50, in a manipulator 100 equipped with the cannula system 600 according to this embodiment, the cannula holder 90 may be fixed to a body insertion port 91, such as a trocar, and forceps drivers 618 connected to the handles 615 of the medical treatment devices 601 may be driven by being controlled by the controller 56.

Next, a cannula system 700 according to an eighth embodiment of the present invention will be described below with reference to the drawings.

The cannula system 700 according to this embodiment is different from the cannula system 600 according to the seventh embodiment with respect to the bent shape of the first tubular members 21 of two cannulas 703, as well as the cannula holder 90.

As shown in FIG. 51, in this embodiment, the first tubular members 21 of the two cannulas 703 are bent in the shape of the letter S. The first tubular member 21 of each of the two cannulas 703 is supported by the cannula holder 90 in a position in which the first tubular member 21 bends away from the other cannula 703 and then bends toward the other cannula 703 from the proximal end toward the distal end. Accordingly, as shown in FIG. 51, the two gripping sections 613 are disposed away from each other when the two handles 615 are positioned closest to each other.

The cannula holder 90 includes an angle changing mechanism 92 that changes the tilt angle of each support member 30 around an axis orthogonal to the direction in which the front bearing 31 and the rear bearing 33, provided in the support member 30 of each cannula 703 supported by the cannula holder 90, are separated from each other.

For example, as shown in FIG. 52, the angle changing mechanism 92 includes two movable bases 94 supported in a pivotable manner around parallel axes relative to a holder base 93, a handle 95 supported by the holder base 93 in a rotatable manner around an axis parallel to the axes of the movable bases 94, a pinion gear 96 fixed to the handle 95, a pair of slide members 97 moved rectilinearly in the direction in which the movable bases 94 are separated from each other, and rack gears 98 that are provided in the slide members 97 and that mesh with the pinion gear 96.

The slide members 97 and the movable bases 94 are connected by inserting pins 99 provided in the movable bases 94 into long holes 97A provided in the slide members 97. Accordingly, as shown in FIG. 53, when the operator rotates the handle 95, the pinion gear 96 rotates, and the rotation of the handle 95 is converted into a rectilinear motion of the slide members 97 by the rack gears 98 meshed with the pinion gear 96, whereby the movable bases 94 are pivoted away from each other.

With the support member 30 of each cannula 703 being fixed to the corresponding movable base 94, the tilt angle of the cannula 703 can be changed by simply rotating the handle 95. As shown in FIG. 54, the support member 30 may be fixed to the movable base 94 in a one-touch operation by fitting a dovetail section 30A provided in the support member 30 into a dovetail groove 94A fixed to the movable base 94.

In the cannula system 700 according to this embodiment, the handle 95 of the cannula holder 90 is operated from a state where the second tubular members 22 of the two cannulas 703 are parallel to each other, as shown in FIG. 51, so that the tilt angle of the support members 30 can be changed in the direction in which the third tubular members 23 of the two cannulas 703 move away from each other, as shown in FIG. 55, whereby an overlapping region (indicated with an oblique line) between the movable ranges of the gripping sections 613 of the two medical treatment device 601 can be increased. Consequently, this is advantageous in that treatment can be performed using the two medical treatment devices 601 over a wide range.

Furthermore, the two gripping sections 613 can be brought close to each other in a state where the handles 615 of the two medical treatment devices 601 are positioned away from each other by the angle changing mechanism 92 of the cannula holder 90. Consequently, when the same target site is to be treated by using the two medical treatment devices 601, interference between the handles 615 can be prevented, thereby preventing reduced workability for the surgeon.

The above-described embodiment also leads to the following aspects.

A first aspect of the present invention provides a cannula including a first tubular member having a first longitudinal axis, a second tubular member having a second longitudinal axis, a connection mechanism that connects the first tubular member and the second tubular member in series, and a rear bearing that supports the second tubular member in a three-dimensionally pivotable manner around an axis intersecting the second longitudinal axis. The first tubular member has a first through-hole into which a flexible and elongated medical treatment device is insertable, and is supported by a trocar in a state where the first tubular member extends through the trocar attached to a body wall of a patient. The second tubular member has a second through-hole into which the medical treatment device is insertable. The connection mechanism connects the first tubular member and the second tubular member in a pivotable manner around an axis orthogonal to a plane including the first longitudinal axis and the second longitudinal axis.

According to this aspect, the first tubular member and the second tubular member connected by the connection mechanism in a pivotable manner around the axis orthogonal to the plane including the first axis and the second axis are supported by the trocar and the rear bearing in a three-dimensionally pivotable manner around the axis intersecting the first longitudinal axis or the second longitudinal axis. Therefore, when the first tubular member and the second tubular member are pivoted around the axis by the connection mechanism, the other end of the first tubular member and the other end of the second tubular member move in the same direction.

Furthermore, the first tubular member and the second tubular member are connected by the connection mechanism with the first through-hole and the second through-hole being disposed in series, so that the medical treatment device can be introduced into the body cavity through the through-holes of the first tubular member and the second tubular member in a state where the first tubular member is supported by the trocar while extending therethrough. Then, the first tubular member and the second tubular member are pivoted around the axis by the connection mechanism, so that the proximal-end operating section and the distal-end treatment section of the medical treatment device can be moved in the same direction. Furthermore, because the proximal-end operating section and the distal-end treatment section are integrated with each other, the proximal-end operating section and the distal-end treatment section can also be moved in the same direction with respect to the axial direction of the medical treatment device.

Accordingly, when the distal-end treatment section inserted into the body cavity is to be directly manipulated by using the proximal-end operating section disposed outside the body cavity, the moving directions of the proximal-end operating section and the distal-end treatment section can be synchronized with each other, so that the surgeon can perform the operation intuitively.

A second aspect of the present invention provides a cannula including a first tubular member having a first longitudinal axis, a second tubular member having a second longitudinal axis, a connection mechanism that connects the first tubular member and the second tubular member in series, a front bearing that supports the first tubular member in a three-dimensionally pivotable manner around an axis intersecting the first longitudinal axis, a rear bearing that supports the second tubular member in a three-dimensionally pivotable manner around an axis intersecting the second longitudinal axis, and a support member that accommodates the connection mechanism therein and that secures a relative position between the front bearing and the rear bearing with a distance therebetween. The first tubular member has a first through-hole into which a flexible and elongated medical treatment device is insertable. The second tubular member has a second through-hole into which the medical treatment device is insertable. The connection mechanism connects the first tubular member and the second tubular member in a pivotable manner around an axis orthogonal to a plane including the first longitudinal axis and the second longitudinal axis.

According to this aspect, the first tubular member and the second tubular member connected by the connection mechanism in a pivotable manner around the axis orthogonal to the plane including the first axis and the second axis are supported by the front bearing and the rear bearing, the relative position between which is secured by the support member, in a three-dimensionally pivotable manner around the axis intersecting the first axis or the second axis. Therefore, when the first tubular member and the second tubular member are pivoted around the axis by the connection mechanism, the ends of the first tubular member and the second tubular member move in the same direction.

Furthermore, the first tubular member and the second tubular member are connected by the connection mechanism with the first through-hole and the second through-hole being disposed in series, so that, by inserting the first tubular member into the body cavity and fixing the support member to the body wall of a patient, the medical treatment device can be introduced into the body cavity through the through-holes of the first tubular member and the second tubular member. Then, the first tubular member and the second tubular member are pivoted around the axis by the connection mechanism, so that the proximal-end operating section and the distal-end treatment section of the medical treatment device can be moved in the same direction. Furthermore, because the proximal-end operating section and the distal-end treatment section are integrated with each other, the proximal-end operating section and the distal-end treatment section can also be moved in the same direction with respect to the axial direction of the medical treatment device.

Accordingly, when the distal-end treatment section inserted into the body cavity is to be directly manipulated by using the proximal-end operating section disposed outside the body cavity, the moving directions of the proximal-end operating section and the distal-end treatment section can be synchronized with each other, so that the surgeon can perform the operation intuitively.

In the above aspect, at least one of the front bearing and the rear bearing may have a spherical bearing structure.

According to this configuration, when the first tubular member or the second tubular member supported by the front bearing or the rear bearing having a spherical bearing structure is pivoted by any angle, the first tubular member or the second tubular member can still be moved with similar low resistance regardless of the pivoting direction and the pivoting angle. Therefore, the medical treatment device can be moved finely.

In the above aspect, at least one of the front bearing and the rear bearing may be fixable after being changed in angle around a pivot axis intersecting the axis.

According to this configuration, the first tubular member and the second tubular member can be pivoted without being limited by the pivoting angle range of the front bearing or the rear bearing.

In the above aspect, the cannula may further include a parallel link extending parallel to a longitudinal direction of the second tubular member and having one end connected to the rear bearing in a three-dimensionally pivotable manner around an axis intersecting an axial direction, a retainer that maintains the parallel link and the second tubular member parallel to each other, and a third tubular member that is elongated and is connected to another end of the parallel link. The third tubular member has a third through-hole into which the medical treatment device is insertable and that is disposed in series relative to the second through-hole. The parallel link may connect the rear bearing and the third tubular member while maintaining axes thereof parallel to each other.

According to this configuration, when the connected area between the first tubular member and the second tubular member is pivoted by the connection mechanism, the third tubular member can be constantly maintained parallel to the axis of the rear bearing. Moreover, the distal-end treatment section of the medical treatment device can be inserted through the third through-hole in the third tubular member and can protrude through the first through-hole in the first tubular member via the second tubular member.

Accordingly, the distal-end treatment section can be pivoted three-dimensionally while the proximal-end operating section of the medical treatment device disposed at the proximal end of the third tubular member is maintained parallel to the axis of the rear bearing. Consequently, the surgeon can manipulate the medical treatment device naturally as if the surgeon is directly holding the distal-end treatment section.

In the above aspect, the connection mechanism may be a cylindrical tube composed of a flexible material.

According to this configuration, the connected area between the first tubular member and the second tubular member can be pivoted by a natural angle due to the connection mechanism, thereby allowing for smoother movement of the medical treatment device. Consequently, the distal-end treatment section can be manipulated more finely. Moreover, cost reduction can be achieved, as compared with a case where the connection mechanism has a component connection structure. Therefore, with the connection mechanism being used singularly, high cost efficiency can be expected when the connection mechanism is to be replaced each time.

In the above aspect, the connection mechanism may have a component connection structure obtained by connecting a plurality of components.

According to this configuration, the medical treatment device to be inserted into the through-holes in the first tubular member and the second tubular member can be pivoted by a large fixed angle. Furthermore, the pivoting angle of the connection mechanism can be increased by increasing the number of components. Accordingly, the medical treatment device can be maintained at a large fixed curvature even with a large pivoting angle. This enables smoother forward and rearward movement of the medical treatment device, so that the medical treatment device can be moved more finely.

In the above aspect, the connection mechanism may have a universal joint structure obtained by connecting connection members that are pivotable around rotation axes intersecting each other.

According to this configuration, the pivoting of the second tubular member can be transmitted to the first tubular member efficiently with high rigidity. Consequently, the movement of the proximal-end operating section of the medical treatment device can be transmitted to the distal-end treatment section with high rigidity, whereby the distal-end treatment section can be manipulated finely.

In the above aspect, the connection mechanism may have a universal joint structure obtained by connecting three or more of the connection members in series.

According to this configuration, the connected area between the first tubular member and the second tubular member can be pivoted by a large angle while maintaining high rigidity. Consequently, the operating range of the medical treatment device can be increased.

In the above aspect, the cannula may further include a driver that causes the medical treatment device inserted in the first tubular member to move forward and rearward in a longitudinal direction and/or rotate around an axis.

According to this configuration, the medical treatment device can be moved forward and rearward or rotated freely by the driver.

In the above aspect, the driver may be accommodated inside the first tubular member.

According to this configuration, the medical treatment device can be moved forward and rearward or rotated directly by the driver.

In the above aspect, the front bearing may include a rotor disposed away from the first tubular member and rotatable around a rotation axis intersecting the axis of the rear bearing, a first link having one end connected to the rotor and extending parallel to the first tubular member, and a pair of second links extending parallel to each other and each having one end connected to the first link and another end connected to the first tubular member. The rotor and the first link, the first link and the second links, and the second links and the first tubular member may be connected to each other in a pivotable manner around a pivot axis orthogonal to the rotation axis of the rotor.

According to this configuration, the first tubular member can be pivoted in the same plane as the first link and the second links by pivoting the first link and the second links around the pivot axis. Moreover, the first tubular member can be pivoted in a plane intersecting the first link and the second links by rotating the rotor around the rotation axis.

In this case, the front bearing is connected to the first tubular member by means of the second links, so that the first tubular member can be supported in a three-dimensionally pivotable manner without having to dispose a bearing structure on the axis of the first tubular member. Accordingly, the first tubular member can be inserted into the body cavity through a normal trocar attached to the body wall of the patient. Moreover, a pivot point where the first tubular member pivots three-dimensionally can be disposed at the center of the body wall of the patient in the thickness direction, so that a procedure with reduced invasiveness can be achieved.

A third aspect of the present invention provides a cannula system including two cannulas according to one of the above aspects and a cannula holder that supports support members in a state where the two cannulas are arranged parallel to each other. The first tubular member of each cannula has a bent shape obtained by bending at least a distal end of the first tubular member toward the other cannula.

According to this aspect, the distal-end treatment sections of the two medical treatment devices can be brought closest to each other in a state where the proximal-end operating sections of the medical treatment devices disposed at the proximal ends of the third tubular members of the two cannulas are positioned away from each other from the position where they are closest to each other. Consequently, when the same target site is to be treated by using the two medical treatment devices, interference between the proximal-end operating sections can be prevented, so that reduced workability for the surgeon can be prevented.

In the above aspect, the cannula holder may include a mechanism that adjusts a tilt angle of each support member around an axis intersecting a direction in which the front bearing and the rear bearing are separated from each other.

According to this configuration, the tilt angles of the support members are adjusted so that the relative tile angle between the two cannulas is changed, whereby the proximal ends of the third tubular members can be moved away from each other. With each first tubular member having a bent shape in which the first tubular member bends away from the other cannula and subsequently bends toward the other cannula, when the relative tilt angle between the two cannula is changed to move the proximal ends of the third tubular members away from each other, the distal-end treatment sections of the two medical treatment devices can be disposed closest to each other. Consequently, when the same target site is to be treated by using the two medical treatment devices, interference between the proximal-end operating sections can be prevented, so that reduced workability for the surgeon can be prevented.

A fourth aspect of the present invention provides a manipulator including the cannula according to one of the above aspects and a medical treatment device. The medical treatment device includes a shaft that is inserted into the first tubular member and the second tubular member and that is bendable by the connection mechanism, a distal-end treatment section that is connected to a distal end of the shaft and that treats an affected site, and a proximal-end operating section that is connected to a proximal end of the shaft and that controls the distal-end treatment section.

According to this aspect, in the cannula, the other end of the first tubular member and the other end of the second tubular member move in the same direction when the connected area between the first tubular member and the second tubular member is bent by the connection mechanism. When the cannula is used for introducing the medical treatment device into the body cavity through the through-holes in the first tubular member and the second tubular member and the distal-end treatment section inserted into the body cavity is to be directly manipulated by using the proximal-end operating section disposed outside the body cavity, the movement of the proximal-end operating section can be synchronized with the movement of the distal-end treatment section, so that the surgeon can perform the operation intuitively.

### REFERENCE SIGNS LIST

1, 601 medical treatment device
3, 103, 203, 303, 403, 503, 603, 703 cannula
11, 611 shaft
13, 613 gripping section (distal-end treatment section)
15, 615 handle (proximal-end operating section)
21 first tubular member
22 second tubular member
23 third tubular member
25, 25A, 25B connection mechanism
29 connection member
30 support member
31 front bearing
33 rear bearing
35 parallel link
39 retainer
55 driver
71 rotor
73 first link
75 second link
90 cannula holder
100 manipulator
600, 700 cannula system

1. A cannula comprising:
a first tubular member having a first longitudinal axis;
a second tubular member having a second longitudinal axis;
a connector that connects the first tubular member and the second tubular member in series; and
a rear bearing that supports the second tubular member in a three-dimensionally pivotable manner around an axis intersecting the second longitudinal axis,
wherein the first tubular member has a first through-hole into which a flexible and elongated medical treatment device is insertable, and is supported by a trocar in a state where the first tubular member extends through the trocar attached to a body wall of a patient,
wherein the second tubular member has a second through-hole into which the medical treatment device is insertable, and
wherein the connector connects the first tubular member and the second tubular member in a pivotable manner around an axis orthogonal to a plane including the first longitudinal axis and the second longitudinal axis.

2. A cannula comprising:
a first tubular member having a first longitudinal axis;
a second tubular member having a second longitudinal axis;
a connection mechanism that connects the first tubular member and the second tubular member in series;
a front bearing that supports the first tubular member in a three-dimensionally pivotable manner around an axis intersecting the first longitudinal axis;
a rear bearing that supports the second tubular member in a three-dimensionally pivotable manner around an axis intersecting the second longitudinal axis; and
a support member configured to accommodate the connection mechanism therein and that secures a relative position between the front bearing and the rear bearing with a distance therebetween,
wherein the first tubular member has a first through-hole into which a flexible and elongated medical treatment device is insertable,
wherein the second tubular member has a second through-hole into which the medical treatment device is insertable, and
wherein the connection mechanism connects the first tubular member and the second tubular member in a pivotable manner around an axis orthogonal to a plane including the first longitudinal axis and the second longitudinal axis.

3. The cannula according to claim 2,
wherein at least one of the front bearing and the rear bearing has a spherical bearing structure.

4. The cannula according to claim 2,
wherein at least one of the front bearing and the rear bearing is fixable after being changed in angle around a pivot axis intersecting the axis.

5. The cannula according to claim 2, further comprising:
a parallel link extending parallel to a longitudinal direction of the second tubular member and having one end connected to the rear bearing in a three-dimensionally pivotable manner around an axis intersecting an axial direction;
a retainer that maintains the parallel link and the second tubular member parallel to each other; and
a third tubular member that is elongated and that is connected to another end of the parallel link, the third tubular member having a third through-hole into which the medical treatment device is insertable and that is disposed in series relative to the second through-hole,
wherein the parallel link connects the rear bearing and the third tubular member while maintaining axes thereof parallel to each other.

6. The cannula according to claim 2,
wherein the connection mechanism is a cylindrical tube composed of a flexible material.

7. The cannula according to claim 2,
wherein the connection mechanism has a component connection structure obtained by connecting a plurality of components.

8. The cannula according to claim 2,
wherein the connection mechanism has a universal joint structure obtained by connecting connection members that are pivotable around rotation axes intersecting each other.

9. The cannula according to claim 8,
wherein the connection mechanism has a universal joint structure obtained by connecting three or more of the connection members in series.

10. The cannula according to claim 2, further comprising:
a driver that causes the medical treatment device inserted in the first tubular member to move forward and rearward in a longitudinal direction and/or rotate around an axis.

11. The cannula according to claim 10,
wherein the driver is accommodated inside the first tubular member.

12. The cannula according to claim 2,
wherein the front bearing comprises
a rotor disposed away from the first tubular member and rotatable around a rotation axis intersecting the axis of the rear bearing,
a first link having one end connected to the rotor and extending parallel to the first tubular member, and
a pair of second links extending parallel to each other and each having one end connected to the first link and another end connected to the first tubular member,
wherein the rotor and the first link, the first link and the second links, and the second links and the first tubular member are connected to each other in a pivotable manner around a pivot axis orthogonal to the rotation axis of the rotor.

13. A cannula system comprising:
two cannulas according to claim 5; and
a cannula holder that supports support members in a state where the two cannulas are arranged parallel to each other,
wherein the first tubular member of each cannula has a bent shape obtained by bending at least a distal end of the first tubular member toward the other cannula.

14. The cannula system according to claim 13,
wherein the cannula holder adjusts a tilt angle of each support member around an axis intersecting a direction in which the front bearing and the rear bearing are separated from each other.

15. A manipulator comprising:
the cannula according to claim 2; and
a medical treatment device including
a shaft that is inserted into the first tubular member and the second tubular member and that is bendable by the connection mechanism,
a gripper that is connected to a distal end of the shaft and that treats an affected site, and
a handle that is connected to a proximal end of the shaft and that controls the gripper.
